SEQUENCE LISTING (44 sequences)

SEQ ID NO: 1 - protein, 50 aa, Araneus diadematus
SGCDVLVQALLEVVSALVSILGSSSIGQINYGASAQYTQMVGQSVAQALA

SEQ ID NO: 2 - protein, 30 aa, Araneus diadematus
SGCDVLVQALLEVVSALVSILGSSSIGQIN

SEQ ID NO: 3 - protein, 21 aa, Araneus diadematus
SGCDVLVQALLEVVSALVSIL

SEQ ID NO: 4 - protein, 1154 aa, Artificial Sequence
  recombinant spider silk protein ADF3KaiLargeNRSH1
MHHHHHHHHHHSSGSSLEVLFQGPARAGSGQQGPGQQGPGQQGPGQQGPYGPGASAAAAAAGGYGPGSGQQGPSQQGPGQQGPGGQGPYGPGASAAAAAAGGYGPGSGQQGPGGQGPYGPGSSAAAAAAGGNGPGSGQQGAGQQGPGQQGPGASAAAAAAGGYGPGSGQQGPGQQGPGGQGPYGPGASAAAAAAGGYGPGSGQGPGQQGPGGQGPYGPGASAAAAAAGGYGPGSGQQGPGQQGPGQQGPGGQGPYGPGASAAAAAAGGYGPGYGQQGPGQQGPGGQGPYGPGASAASAASGGYGPGSGQQGPGQQGPGGQGPYGPGASAAAAAAGGYGPGSGQQGPGQQGPGQQGPGQQGPGGQGPYGPGASAAAAAAGGYGPGSGQQGPGQQGPGQQGPGQQGPGQQGPGQQGPGQQGPGQQGPGQQGPGGQGAYGPGASAAAGAAGGYGPGSGQQGPGQQGPGQQGPGQQGPGQQGPGQQGPGQQGPGQQGPYGPGASAAAAAAGGYGPGSGQQGPGQQGPGQQGPGGQGPYGPGAASAAVSVSRARAGSGQQGPGQQGPGQQGPGQQGPYGPGASAAAAAAGGYGPGSGQQGPSQQGPGQQGPGGQGPYGPGASAAAAAAGGYGPGSGQQGPGGQGPYGPGSSAAAAAAGGNGPGSGQQGAGQQGPGQQGPGASAAAAAAGGYGPGSGQQGPGQQGPGGQGPYGPGASAAAAAAGGYGPGSGQGPGQQGPGGQGPYGPGASAAAAAAGGYGPGSGQQGPGQQGPGQQGPGGQGPYGPGASAAAAAAGGYGPGYGQQGPGQQGPGGQGPYGPGASAASAASGGYGPGSGQQGPGQQGPGGQGPYGPGASAAAAAAGGYGPGSGQQGPGQQGPGQQGPGQQGPGGQGPYGPGASAAAAAAGGYGPGSGQQGPGQQGPGQQGPGQQGPGQQGPGQQGPGQQGPGQQGPGQQGPGGQGAYGPGASAAAGAAGGYGPGSGQQGPGQQGPGQQGPGQQGPGQQGPGQQGPGQQGPGQQGPYGPGASAAAAAAGGYGPGSGQQGPGQQGPGQQGPGGQGPYGPGAASAAVSVGGYGPQSSSVPVASAVASRLSSPAASSRVSSAVSSLVSSGPTKHAALSNTISSVVSQVSASNPGLSGCDVLVQALLEVVSALVSIL

SEQ ID NO: 5 - protein, 24 aa, Artificial Sequence
  His tag and start codon
MHHHHHHHHHHSSGSSLEVLFQGP

SEQ ID NO: 6 - protein, 597 aa, Artificial Sequence
  Met-PRT380
MGPGQQGPYGPGASAAAAAGQNGPGSGQQGPGQSAAAAAGQYGPGQQGPGQQGPGSSAAAAAGPGQYGPGQQGPSASAAAAAGPGSGQQGPGASAAAAAGQYGPGQQGPGQQGPGSSAAAAAGQYGSGPGQQGPYGSAAAAAGPGSGQYGQGPYGPGASAAAAAGPGQYGPGQQGPSASAAAAAGSGQQGPGQYGPYASAAAAAGQYGSGPGQQGPYGPGQSAAAAAGSGQQGPGQQGPYASAAAAAGPGQQGPYGPGSSAAAAAGQYGYGPGQQGPYGPGASAAAAAGQNGPGSGQYGPGQQGPGQSAAAAAGPGQQGPYGPGASAAAAAGQYGPGQQGPGQYGPGSSAAAAAGPGQQGPYGPGSSAAAAAGQYGPGQQGPYGPGQSAAAAAGQYQQGPGQQGPYGPGASAAAAAGPGQQGPYGPGASAAAAAGPGQYGPGQQGPSASAAAAAGQYGSGPGQYGPYGPGQSAAAAAGPGSGQQGQGPYGPGASAAAAAGQYGPGQQGPYGPGQSAAAAAGPGSGQYGPGASAAAAAGQNGPGSGQYGPGQQGPGQSAAAAAGQYQQGPGQQGPYGPGASAAAAAGPGSGQQGPGAS

SEQ ID NO: 7 - protein, 590 aa, Artificial Sequence
  Met-PRT410
MGPGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGAS

SEQ ID NO: 8 - protein, 565 aa, Artificial Sequence
  Met-PRT525
MGPGQQGPYGPGASAAAAAAAGSNGPGSGQQGPGQSGQYGPGQQGPGQQGPGSSAAAAAAAGPGQYGPGQQGPSASAAAAAAAGPGSGQQGPGASGQYGPGQQGPGQQGPGSSAAAAAAAGSYGSGPGQQGPYGSAAAAAAAGPGSGQYGQGPYGPGASGPGQYGPGQQGPSASAAAAAAAGSGQQGPGQYGPYASAAAAAAAGSYGSGPGQQGPYGPGQSGSGQQGPGQQGPYASAAAAAAAGPGQQGPYGPGSSAAAAAAAGSYGYGPGQQGPYGPGASGQNGPGSGQYGPGQQGPGPSAAAAAAAGPGQQGPYGPGASAAAAAAAGSYGPGQQGPGQYGPGSSGPGQQGPYGPGSSAAAAAAAGSYGPGQQGPYGPGPSAAAAAAAGSYQQGPGQQGPYGPGASGPGQQGPYGPGASAAAAAAAGPGQYGPGQQGPSASAAAAAAAGSYGSGPGQYGPYGPGQSGPGSGQQGQGPYGPGASAAAAAAAGSYGPGQQGPYGPGPSAAAAAAAGPGSGQYGPGASGQNGPGSGQYGPGQQGPGPSAAAAAAAGPGSGQQGPGAS

SEQ ID NO: 9 - protein, 2364 aa, Artificial Sequence
  Met-PRT799
MGPGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGASGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGASGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGASGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGSSVDKLAAALEHHHHHH

SEQ ID NO: 10 - protein, 597 aa, Artificial Sequence
  Met-PRT313
MGPGGQGPYGPGASAAAAAGGNGPGSGQQGPGGSAAAAAGGYGPGGQGPGQQGPGSSAAAAAGPGGYGPGGQGPSASAAAAAGPGSGQQGPGASAAAAAGGYGPGGQGPGQQGPGSSAAAAAGGYGSGPGQQGPYGSAAAAAGPGSGGYGQGPYGPGASAAAAAGPGGYGPGGQGPSASAAAAAGSGQQGPGGYGPYASAAAAAGGYGSGPGQQGPYGPGGSAAAAAGSGQQGPGQQGPYASAAAAAGPGGQGPYGPGSSAAAAAGGYGYGPGGQGPYGPGASAAAAAGGNGPGSGGYGPGQQGPGGSAAAAAGPGGQGPYGPGASAAAAAGGYGPGGQGPGGYGPGSSAAAAAGPGGQGPYGPGSSAAAAAGGYGPGQQGPYGPGGSAAAAAGGYQQGPGGQGPYGPGASAAAAAGPGGQGPYGPGASAAAAAGPGGYGPGGQGPSASAAAAAGGYGSGPGGYGPYGPGGSAAAAAGPGSGQQGQGPYGPGASAAAAAGGYGPGQQGPYGPGGSAAAAAGPGSGGYGPGASAAAAAGGNGPGSGGYGPGQQGPGGSAAAAAGGYQQGPGGQGPYGPGASAAAAAGPGSGQQGPGAS

SEQ ID NO: 11 - protein, 12 aa, Artificial Sequence
  HisTag
MHHHHHHSSGSS

SEQ ID NO: 12 - protein, 608 aa, Artificial Sequence
  PRT380
MHHHHHHSSGSSGPGQQGPYGPGASAAAAAGQNGPGSGQQGPGQSAAAAAGQYGPGQQGPGQQGPGSSAAAAAGPGQYGPGQQGPSASAAAAAGPGSGQQGPGASAAAAAGQYGPGQQGPGQQGPGSSAAAAAGQYGSGPGQQGPYGSAAAAAGPGSGQYGQGPYGPGASAAAAAGPGQYGPGQQGPSASAAAAAGSGQQGPGQYGPYASAAAAAGQYGSGPGQQGPYGPGQSAAAAAGSGQQGPGQQGPYASAAAAAGPGQQGPYGPGSSAAAAAGQYGYGPGQQGPYGPGASAAAAAGQNGPGSGQYGPGQQGPGQSAAAAAGPGQQGPYGPGASAAAAAGQYGPGQQGPGQYGPGSSAAAAAGPGQQGPYGPGSSAAAAAGQYGPGQQGPYGPGQSAAAAAGQYQQGPGQQGPYGPGASAAAAAGPGQQGPYGPGASAAAAAGPGQYGPGQQGPSASAAAAAGQYGSGPGQYGPYGPGQSAAAAAGPGSGQQGQGPYGPGASAAAAAGQYGPGQQGPYGPGQSAAAAAGPGSGQYGPGASAAAAAGQNGPGSGQYGPGQQGPGQSAAAAAGQYQQGPGQQGPYGPGASAAAAAGPGSGQQGPGAS

SEQ ID NO: 13 - protein, 601 aa, Artificial Sequence
  PRT410
MHHHHHHSSGSSGPGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGAS

SEQ ID NO: 14 - protein, 576 aa, Artificial Sequence
  PRT525
MHHHHHHSSGSSGPGQQGPYGPGASAAAAAAAGSNGPGSGQQGPGQSGQYGPGQQGPGQQGPGSSAAAAAAAGPGQYGPGQQGPSASAAAAAAAGPGSGQQGPGASGQYGPGQQGPGQQGPGSSAAAAAAAGSYGSGPGQQGPYGSAAAAAAAGPGSGQYGQGPYGPGASGPGQYGPGQQGPSASAAAAAAAGSGQQGPGQYGPYASAAAAAAAGSYGSGPGQQGPYGPGQSGSGQQGPGQQGPYASAAAAAAAGPGQQGPYGPGSSAAAAAAAGSYGYGPGQQGPYGPGASGQNGPGSGQYGPGQQGPGPSAAAAAAAGPGQQGPYGPGASAAAAAAAGSYGPGQQGPGQYGPGSSGPGQQGPYGPGSSAAAAAAAGSYGPGQQGPYGPGPSAAAAAAAGSYQQGPGQQGPYGPGASGPGQQGPYGPGASAAAAAAAGPGQYGPGQQGPSASAAAAAAAGSYGSGPGQYGPYGPGQSGPGSGQQGQGPYGPGASAAAAAAAGSYGPGQQGPYGPGPSAAAAAAAGPGSGQYGPGASGQNGPGSGQYGPGQQGPGPSAAAAAAAGPGSGQQGPGAS

SEQ ID NO: 15 - protein, 2375 aa, Artificial Sequence
  PRT799
MHHHHHHSSGSSGPGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGASGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGASGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGASGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGSSVDKLAAALEHHHHHH

SEQ ID NO: 16 - protein, 608 aa, Artificial Sequence
  PRT313
MHHHHHHSSGSSGPGGQGPYGPGASAAAAAGGNGPGSGQQGPGGSAAAAAGGYGPGGQGPGQQGPGSSAAAAAGPGGYGPGGQGPSASAAAAAGPGSGQQGPGASAAAAAGGYGPGGQGPGQQGPGSSAAAAAGGYGSGPGQQGPYGSAAAAAGPGSGGYGQGPYGPGASAAAAAGPGGYGPGGQGPSASAAAAAGSGQQGPGGYGPYASAAAAAGGYGSGPGQQGPYGPGGSAAAAAGSGQQGPGQQGPYASAAAAAGPGGQGPYGPGSSAAAAAGGYGYGPGGQGPYGPGASAAAAAGGNGPGSGGYGPGQQGPGGSAAAAAGPGGQGPYGPGASAAAAAGGYGPGGQGPGGYGPGSSAAAAAGPGGQGPYGPGSSAAAAAGGYGPGQQGPYGPGGSAAAAAGGYQQGPGGQGPYGPGASAAAAAGPGGQGPYGPGASAAAAAGPGGYGPGGQGPSASAAAAAGGYGSGPGGYGPYGPGGSAAAAAGPGSGQQGQGPYGPGASAAAAAGGYGPGQQGPYGPGGSAAAAAGPGSGGYGPGASAAAAAGGNGPGSGGYGPGQQGPGGSAAAAAGGYQQGPGGQGPYGPGASAAAAAGPGSGQQGPGAS

SEQ ID NO: 17 - protein, 590 aa, Artificial Sequence
  Met-PRT399
MGPGGQGPYGPGASAAAAAGGNGPGSGQQGPGGSGGYGPGGQGPGQQGPGSSAAAAAGPGGYGPGGQGPSASAAAAAGPGSGQQGPGASGGYGPGGQGPGQQGPGSSAAAAAGGYGSGPGQQGPYGSAAAAAGPGSGGYGQGPYGPGASGPGGYGPGGQGPSASAAAAAGSGQQGPGGYGPYASAAAAAGGYGSGPGQQGPYGPGGSGSGQQGPGQQGPYASAAAAAGPGGQGPYGPGSSAAAAAGGYGYGPGGQGPYGPGASGGNGPGSGGYGPGQQGPGGSAAAAAGPGGQGPYGPGASAAAAAGGYGPGGQGPGGYGPGSSGPGGQGPYGPGSSAAAAAGGYGPGQQGPYGPGGSAAAAAGGYQQGPGGQGPYGPGASGPGGQGPYGPGASAAAAAGPGGYGPGGQGPSASAAAAAGGYGSGPGGYGPYGPGGSGPGSGQQGQGPYGPGASAAAAAGGYGPGQQGPYGPGGSAAAAAGPGSGGYGPGASGGNGPGSGGYGPGQQGPGGSAAAAAGGYQQGPGGQGPYGPGASAAAAAGGYGSGPGQQGPYGPGGSGSGQQGPGQQGPYASAAAAAGPGSGQQGPGAS

SEQ ID NO: 18 - protein, 601 aa, Artificial Sequence
  PRT399
MHHHHHHSSGSSGPGGQGPYGPGASAAAAAGGNGPGSGQQGPGGSGGYGPGGQGPGQQGPGSSAAAAAGPGGYGPGGQGPSASAAAAAGPGSGQQGPGASGGYGPGGQGPGQQGPGSSAAAAAGGYGSGPGQQGPYGSAAAAAGPGSGGYGQGPYGPGASGPGGYGPGGQGPSASAAAAAGSGQQGPGGYGPYASAAAAAGGYGSGPGQQGPYGPGGSGSGQQGPGQQGPYASAAAAAGPGGQGPYGPGSSAAAAAGGYGYGPGGQGPYGPGASGGNGPGSGGYGPGQQGPGGSAAAAAGPGGQGPYGPGASAAAAAGGYGPGGQGPGGYGPGSSGPGGQGPYGPGSSAAAAAGGYGPGQQGPYGPGGSAAAAAGGYQQGPGGQGPYGPGASGPGGQGPYGPGASAAAAAGPGGYGPGGQGPSASAAAAAGGYGSGPGGYGPYGPGGSGPGSGQQGQGPYGPGASAAAAAGGYGPGQQGPYGPGGSAAAAAGPGSGGYGPGASGGNGPGSGGYGPGQQGPGGSAAAAAGGYQQGPGGQGPYGPGASAAAAAGGYGSGPGQQGPYGPGGSGSGQQGPGQQGPYASAAAAAGPGSGQQGPGAS

SEQ ID NO: 19 - protein, 612 aa, Artificial Sequence
  Met-PRT720
MGPGQQGPYGPGASAAAAAGQNGPGSGQQGPGQSGQYGPGQQGPGQQGPGSSAAAAAGPGQYVLIGPGQQVLIGPSASAAAAAGPGSGQQGPGASGQYGPGQQGPGQQGPGSSAAAAAGSYGSVLIGPGQQVLIGPYGSAAAAAGPGSGQYGQGPYGPGASGPGQYGPGQQGPSASAAAAAGSGQQVLIGPGQYVLIGPYASAAAAAGQYGSGPGQQGPYGPGQSGSGQQGPGQQGPYASAAAAAGPGQQVLIGPYVLIGPGSSAAAAAGQYGYGPGQQGPYGPGASGQNGPGSGQYGPGQQGPGQSAAAAAGPGQQVLIGPYVLIGPGASAAAAAGQYGPGQQGPGQYGPGSSGPGQQGPYGPGSSAAAAAGSYGPGQQVLIGPYVLIGPGPSAAAAAGQYQQGPGQQGPYGPGASGPGQQGPYGPGASAAAAAGPGQYVLIGPGQQVLIGPSASAAAAAGQYGSGPGQYGPYGPGQSGPGSGQQGQGPYGPGASAAAAAGSYGPGQQVLIGPYVLIGPGPSAAAAAGPGSGQYGPGASGQNGPGSGQYGPGQQGPGQSAAAAAGQYQQVLIGPGQQGPYVLIGPGASAAAAAGPGSGQQVLIGPGASVLI

SEQ ID NO: 20 - protein, 592 aa, Artificial Sequence
  Met-PRT665
MGPGQQGPYGPGASAAAAAAAGSNGPGSGQQGPGQSGQYGPGQQGPGQQGPGSSAAAAAAAGPGQYVLIGPGQQGPSASAAAAAAAGPGSGQQGPGASGQYGPGQQGPGQQGPGSSAAAAAAAGSYGSVLIGPGQQGPYGSAAAAAAAGPGSGQYGQGPYGPGASGPGQYGPGQQGPSASAAAAAAAGSGQQVLIGPGQYGPYASAAAAAAAGSYGSGPGQQGPYGPGQSGSGQQGPGQQGPYASAAAAAAAGPGQQVLIGPYGPGSSAAAAAAAGSYGYGPGQQGPYGPGASGQNGPGSGQYGPGQQGPGPSAAAAAAAGPGQQVLIGPYGPGASAAAAAAAGSYGPGQQGPGQYGPGSSGPGQQGPYGPGSSAAAAAAAGSYGPGQQVLIGPYGPGPSAAAAAAAGSYQQGPGQQGPYGPGASGPGQQGPYGPGASAAAAAAAGPGQYVLIGPGQQGPSASAAAAAAAGSYGSGPGQYGPYGPGQSGPGSGQQGQGPYGPGASAAAAAAAGSYGPGQQVLIGPYGPGPSAAAAAAAGPGSGQYGPGASGQNGPGSGQYGPGQQGPGPSAAAAAAAGPGSGQQGPGASVLI

SEQ ID NO: 21 - protein, 619 aa, Artificial Sequence
  Met-PRT666
MGPGQQGPYGPGASAAAAAAAGSNGPGSGQQGPGQSGQYGPGQQGPGQQGPGSSAAAAAAAGPGQYVLIGPGQQVLIGPSASAAAAAAAGPGSGQQGPGASGQYGPGQQGPGQQGPGSSAAAAAAAGSYGSVLIGPGQQVLIGPYGSAAAAAAAGPGSGQYGQGPYGPGASGPGQYGPGQQGPSASAAAAAAAGSGQQVLIGPGQYVLIGPYASAAAAAAAGSYGSGPGQQGPYGPGQSGSGQQGPGQQGPYASAAAAAAAGPGQQVLIGPYVLIGPGSSAAAAAAAGSYGYGPGQQGPYGPGASGQNGPGSGQYGPGQQGPGPSAAAAAAAGPGQQVLIGPYVLIGPGASAAAAAAAGSYGPGQQGPGQYGPGSSGPGQQGPYGPGSSAAAAAAAGSYGPGQQVLIGPYVLIGPGPSAAAAAAAGSYQQGPGQQGPYGPGASGPGQQGPYGPGASAAAAAAAGPGQYVLIGPGQQVLIGPSASAAAAAAAGSYGSGPGQYGPYGPGQSGPGSGQQGQGPYGPGASAAAAAAAGSYGPGQQVLIGPYVLIGPGPSAAAAAAAGPGSGQYGPGASGQNGPGSGQYGPGQQGPGPSAAAAAAAGPGSGQQVLIGPGASVLI

SEQ ID NO: 22 - protein, 623 aa, Artificial Sequence
  PRT720
MHHHHHHSSGSSGPGQQGPYGPGASAAAAAGQNGPGSGQQGPGQSGQYGPGQQGPGQQGPGSSAAAAAGPGQYVLIGPGQQVLIGPSASAAAAAGPGSGQQGPGASGQYGPGQQGPGQQGPGSSAAAAAGSYGSVLIGPGQQVLIGPYGSAAAAAGPGSGQYGQGPYGPGASGPGQYGPGQQGPSASAAAAAGSGQQVLIGPGQYVLIGPYASAAAAAGQYGSGPGQQGPYGPGQSGSGQQGPGQQGPYASAAAAAGPGQQVLIGPYVLIGPGSSAAAAAGQYGYGPGQQGPYGPGASGQNGPGSGQYGPGQQGPGQSAAAAAGPGQQVLIGPYVLIGPGASAAAAAGQYGPGQQGPGQYGPGSSGPGQQGPYGPGSSAAAAAGSYGPGQQVLIGPYVLIGPGPSAAAAAGQYQQGPGQQGPYGPGASGPGQQGPYGPGASAAAAAGPGQYVLIGPGQQVLIGPSASAAAAAGQYGSGPGQYGPYGPGQSGPGSGQQGQGPYGPGASAAAAAGSYGPGQQVLIGPYVLIGPGPSAAAAAGPGSGQYGPGASGQNGPGSGQYGPGQQGPGQSAAAAAGQYQQVLIGPGQQGPYVLIGPGASAAAAAGPGSGQQVLIGPGASVLI

SEQ ID NO: 23 - protein, 603 aa, Artificial Sequence
  PRT665
MHHHHHHSSGSSGPGQQGPYGPGASAAAAAAAGSNGPGSGQQGPGQSGQYGPGQQGPGQQGPGSSAAAAAAAGPGQYVLIGPGQQGPSASAAAAAAAGPGSGQQGPGASGQYGPGQQGPGQQGPGSSAAAAAAAGSYGSVLIGPGQQGPYGSAAAAAAAGPGSGQYGQGPYGPGASGPGQYGPGQQGPSASAAAAAAAGSGQQVLIGPGQYGPYASAAAAAAAGSYGSGPGQQGPYGPGQSGSGQQGPGQQGPYASAAAAAAAGPGQQVLIGPYGPGSSAAAAAAAGSYGYGPGQQGPYGPGASGQNGPGSGQYGPGQQGPGPSAAAAAAAGPGQQVLIGPYGPGASAAAAAAAGSYGPGQQGPGQYGPGSSGPGQQGPYGPGSSAAAAAAAGSYGPGQQVLIGPYGPGPSAAAAAAAGSYQQGPGQQGPYGPGASGPGQQGPYGPGASAAAAAAAGPGQYVLIGPGQQGPSASAAAAAAAGSYGSGPGQYGPYGPGQSGPGSGQQGQGPYGPGASAAAAAAAGSYGPGQQVLIGPYGPGPSAAAAAAAGPGSGQYGPGASGQNGPGSGQYGPGQQGPGPSAAAAAAAGPGSGQQGPGASVLI

SEQ ID NO: 24 - protein, 630 aa, Artificial Sequence
  PRT666
MHHHHHHSSGSSGPGQQGPYGPGASAAAAAAAGSNGPGSGQQGPGQSGQYGPGQQGPGQQGPGSSAAAAAAAGPGQYVLIGPGQQVLIGPSASAAAAAAAGPGSGQQGPGASGQYGPGQQGPGQQGPGSSAAAAAAAGSYGSVLIGPGQQVLIGPYGSAAAAAAAGPGSGQYGQGPYGPGASGPGQYGPGQQGPSASAAAAAAAGSGQQVLIGPGQYVLIGPYASAAAAAAAGSYGSGPGQQGPYGPGQSGSGQQGPGQQGPYASAAAAAAAGPGQQVLIGPYVLIGPGSSAAAAAAAGSYGYGPGQQGPYGPGASGQNGPGSGQYGPGQQGPGPSAAAAAAAGPGQQVLIGPYVLIGPGASAAAAAAAGSYGPGQQGPGQYGPGSSGPGQQGPYGPGSSAAAAAAAGSYGPGQQVLIGPYVLIGPGPSAAAAAAAGSYQQGPGQQGPYGPGASGPGQQGPYGPGASAAAAAAAGPGQYVLIGPGQQVLIGPSASAAAAAAAGSYGSGPGQYGPYGPGQSGPGSGQQGQGPYGPGASAAAAAAAGSYGPGQQVLIGPYVLIGPGPSAAAAAAAGPGSGQYGPGASGQNGPGSGQYGPGQQGPGPSAAAAAAAGPGSGQQVLIGPGASVLI

SEQ ID NO: 25 - protein, 593 aa, Artificial Sequence
  Met-PRT888
MGSSGPGVLGPYGPGASAAAAAGQNGPGSGVLGPGQSGQYGPGVLGPGVLGPGSSAAAAAGPGQYGPGVLGPSASAAAAAGPGSGVLGPGASGQYGPGVLGPGVLGPGSSAAAAAGQYGSGPGVLGPYGSAAAAAGPGSGQYGQGPYGPGASGPGQYGPGVLGPSASAAAAAGSGVLGPGQYGPYASAAAAAGQYGSGPGVLGPYGPGQSGSGVLGPGVLGPYASAAAAAGPGVLGPYGPGSSAAAAAGQYGYGPGVLGPYGPGASGQNGPGSGQYGPGVLGPGQSAAAAAGPGVLGPYGPGASAAAAAGQYGPGVLGPGQYGPGSSGPGVLGPYGPGSSAAAAAGQYGPGVLGPYGPGQSAAAAAGQYVLGPGVLGPYGPGASGPGVLGPYGPGASAAAAAGPGQYGPGVLGPSASAAAAAGQYGSGPGQYGPYGPGQSGPGSGVLGQGPYGPGASAAAAAGQYGPGVLGPYGPGQSAAAAAGPGSGQYGPGASGQNGPGSGQYGPGVLGPGQSAAAAAGQYVLGPGVLGPYGPGASAAAAAGQYGSGPGVLGPYGPGQSGSGVLGPGVLGPYASAAAAAGPGSGVLGPGAS

SEQ ID NO: 26 - protein, 590 aa, Artificial Sequence
  Met-PRT965
MGPGTSGPYGPGASAAAAAGANGPGSGTSGPGASGAYGPGTSGPGTSGPGSSAAAAAGPGAYGPGTSGPSASAAAAAGPGSGTSGPGASGAYGPGTSGPGTSGPGSSAAAAAGAYGSGPGTSGPYGSAAAAAGPGSGAYGAGPYGPGASGPGAYGPGTSGPSASAAAAAGSGTSGPGAYGPYASAAAAAGAYGSGPGTSGPYGPGASGSGTSGPGTSGPYASAAAAAGPGTSGPYGPGSSAAAAAGAYGYGPGTSGPYGPGASGANGPGSGAYGPGTSGPGASAAAAAGPGTSGPYGPGASAAAAAGAYGPGTSGPGAYGPGSSGPGTSGPYGPGSSAAAAAGAYGPGTSGPYGPGASAAAAAGAYTSGPGTSGPYGPGASGPGTSGPYGPGASAAAAAGPGAYGPGTSGPSASAAAAAGAYGSGPGAYGPYGPGASGPGSGTSGAGPYGPGASAAAAAGAYGPGTSGPYGPGASAAAAAGPGSGAYGPGASGANGPGSGAYGPGTSGPGASAAAAAGAYTSGPGTSGPYGPGASAAAAAGAYGSGPGTSGPYGPGASGSGTSGPGTSGPYASAAAAAGPGSGTSGPGAS

SEQ ID NO: 27 - protein, 593 aa, Artificial Sequence
  Met-PRT889
MGSSGPGVLGPYGPGASAAAAAGINGPGSGVLGPGISGIYGPGVLGPGVLGPGSSAAAAAGPGIYGPGVLGPSASAAAAAGPGSGVLGPGASGIYGPGVLGPGVLGPGSSAAAAAGIYGSGPGVLGPYGSAAAAAGPGSGIYGIGPYGPGASGPGIYGPGVLGPSASAAAAAGSGVLGPGIYGPYASAAAAAGIYGSGPGVLGPYGPGISGSGVLGPGVLGPYASAAAAAGPGVLGPYGPGSSAAAAAGIYGYGPGVLGPYGPGASGINGPGSGIYGPGVLGPGISAAAAAGPGVLGPYGPGASAAAAAGIYGPGVLGPGIYGPGSSGPGVLGPYGPGSSAAAAAGIYGPGVLGPYGPGISAAAAAGIYVLGPGVLGPYGPGASGPGVLGPYGPGASAAAAAGPGIYGPGVLGPSASAAAAAGIYGSGPGIYGPYGPGISGPGSGVLGIGPYGPGASAAAAAGIYGPGVLGPYGPGISAAAAAGPGSGIYGPGASGINGPGSGIYGPGVLGPGISAAAAAGIYVLGPGVLGPYGPGASAAAAAGIYGSGPGVLGPYGPGISGSGVLGPGVLGPYASAAAAAGPGSGVLGPGAS

SEQ ID NO: 28 - protein, 590 aa, Artificial Sequence
  Met-PRT916
MGPGVIGPYGPGASAAAAAGLNGPGSGVIGPGLSGLYGPGVIGPGVIGPGSSAAAAAGPGLYGPGVIGPSASAAAAAGPGSGVIGPGASGLYGPGVIGPGVIGPGSSAAAAAGLYGSGPGVIGPYGSAAAAAGPGSGLYGLGPYGPGASGPGLYGPGVIGPSASAAAAAGSGVIGPGLYGPYASAAAAAGLYGSGPGVIGPYGPGLSGSGVIGPGVIGPYASAAAAAGPGVIGPYGPGSSAAAAAGLYGYGPGVIGPYGPGASGLNGPGSGLYGPGVIGPGLSAAAAAGPGVIGPYGPGASAAAAAGLYGPGVIGPGLYGPGSSGPGVIGPYGPGSSAAAAAGLYGPGVIGPYGPGLSAAAAAGLYVIGPGVIGPYGPGASGPGVIGPYGPGASAAAAAGPGLYGPGVIGPSASAAAAAGLYGSGPGLYGPYGPGLSGPGSGVIGLGPYGPGASAAAAAGLYGPGVIGPYGPGLSAAAAAGPGSGLYGPGASGLNGPGSGLYGPGVIGPGLSAAAAAGLYVIGPGVIGPYGPGASAAAAAGLYGSGPGVIGPYGPGLSGSGVIGPGVIGPYASAAAAAGPGSGVIGPGAS

SEQ ID NO: 29 - protein, 590 aa, Artificial Sequence
  Met-PRT918
MGPGVFGPYGPGASAAAAAGINGPGSGVFGPGISGIYGPGVFGPGVFGPGSSAAAAAGPGIYGPGVFGPSASAAAAAGPGSGVFGPGASGIYGPGVFGPGVFGPGSSAAAAAGIYGSGPGVFGPYGSAAAAAGPGSGIYGIGPYGPGASGPGIYGPGVFGPSASAAAAAGSGVFGPGIYGPYASAAAAAGIYGSGPGVFGPYGPGISGSGVFGPGVFGPYASAAAAAGPGVFGPYGPGSSAAAAAGIYGYGPGVFGPYGPGASGINGPGSGIYGPGVFGPGISAAAAAGPGVFGPYGPGASAAAAAGIYGPGVFGPGIYGPGSSGPGVFGPYGPGSSAAAAAGIYGPGVFGPYGPGISAAAAAGIYVFGPGVFGPYGPGASGPGVFGPYGPGASAAAAAGPGIYGPGVFGPSASAAAAAGIYGSGPGIYGPYGPGISGPGSGVFGIGPYGPGASAAAAAGIYGPGVFGPYGPGISAAAAAGPGSGIYGPGASGINGPGSGIYGPGVFGPGISAAAAAGIYVFGPGVFGPYGPGASAAAAAGIYGSGPGVFGPYGPGISGSGVFGPGVFGPYASAAAAAGPGSGVFGPGAS

SEQ ID NO: 30 - protein, 565 aa, Artificial Sequence
  Met-PRT699
MGPGVLGPYGPGASAAAAAAAGSNGPGSGVLGPGQSGQYGPGVLGPGVLGPGSSAAAAAAAGPGQYGPGVLGPSASAAAAAAAGPGSGVLGPGASGQYGPGVLGPGVLGPGSSAAAAAAAGSYGSGPGVLGPYGSAAAAAAAGPGSGQYGQGPYGPGASGPGQYGPGVLGPSASAAAAAAAGSGVLGPGQYGPYASAAAAAAAGSYGSGPGVLGPYGPGQSGSGVLGPGVLGPYASAAAAAAAGPGVLGPYGPGSSAAAAAAAGSYGYGPGVLGPYGPGASGQNGPGSGQYGPGVLGPGPSAAAAAAAGPGVLGPYGPGASAAAAAAAGSYGPGVLGPGQYGPGSSGPGVLGPYGPGSSAAAAAAAGSYGPGVLGPYGPGPSAAAAAAAGSYVLGPGVLGPYGPGASGPGVLGPYGPGASAAAAAAAGPGQYGPGVLGPSASAAAAAAAGSYGSGPGQYGPYGPGQSGPGSGVLGQGPYGPGASAAAAAAAGSYGPGVLGPYGPGPSAAAAAAAGPGSGQYGPGASGQNGPGSGQYGPGVLGPGPSAAAAAAAGPGSGVLGPGAS

SEQ ID NO: 31 - protein, 565 aa, Artificial Sequence
  Met-PRT698
MGPGVLGPYGPGASAAAAAAAGSNGPGSGVLGPGISGIYGPGVLGPGVLGPGSSAAAAAAAGPGIYGPGVLGPSASAAAAAAAGPGSGVLGPGASGIYGPGVLGPGVLGPGSSAAAAAAAGSYGSGPGVLGPYGSAAAAAAAGPGSGIYGIGPYGPGASGPGIYGPGVLGPSASAAAAAAAGSGVLGPGIYGPYASAAAAAAAGSYGSGPGVLGPYGPGISGSGVLGPGVLGPYASAAAAAAAGPGVLGPYGPGSSAAAAAAAGSYGYGPGVLGPYGPGASGINGPGSGIYGPGVLGPGPSAAAAAAAGPGVLGPYGPGASAAAAAAAGSYGPGVLGPGIYGPGSSGPGVLGPYGPGSSAAAAAAAGSYGPGVLGPYGPGPSAAAAAAAGSYVLGPGVLGPYGPGASGPGVLGPYGPGASAAAAAAAGPGIYGPGVLGPSASAAAAAAAGSYGSGPGIYGPYGPGISGPGSGVLGIGPYGPGASAAAAAAAGSYGPGVLGPYGPGPSAAAAAAAGPGSGIYGPGASGINGPGSGIYGPGVLGPGPSAAAAAAAGPGSGVLGPGAS

SEQ ID NO: 32 - protein, 1179 aa, Artificial Sequence
  Met-PRT966
MGPGVFGPYGPGASAAAAAGINGPGSGVFGPGISGIYGPGVFGPGVFGPGSSAAAAAGPGIYGPGVFGPSASAAAAAGPGSGVFGPGASGIYGPGVFGPGVFGPGSSAAAAAGIYGSGPGVFGPYGSAAAAAGPGSGIYGIGPYGPGASGPGIYGPGVFGPSASAAAAAGSGVFGPGIYGPYASAAAAAGIYGSGPGVFGPYGPGISGSGVFGPGVFGPYASAAAAAGPGVFGPYGPGSSAAAAAGIYGYGPGVFGPYGPGASGINGPGSGIYGPGVFGPGISAAAAAGPGVFGPYGPGASAAAAAGIYGPGVFGPGIYGPGSSGPGVFGPYGPGSSAAAAAGIYGPGVFGPYGPGISAAAAAGIYVFGPGVFGPYGPGASGPGVFGPYGPGASAAAAAGPGIYGPGVFGPSASAAAAAGIYGSGPGIYGPYGPGISGPGSGVFGIGPYGPGASAAAAAGIYGPGVFGPYGPGISAAAAAGPGSGIYGPGASGINGPGSGIYGPGVFGPGISAAAAAGIYVFGPGVFGPYGPGASAAAAAGIYGSGPGVFGPYGPGISGSGVFGPGVFGPYASAAAAAGPGSGVFGPGASGPGVFGPYGPGASAAAAAGINGPGSGVFGPGISGIYGPGVFGPGVFGPGSSAAAAAGPGIYGPGVFGPSASAAAAAGPGSGVFGPGASGIYGPGVFGPGVFGPGSSAAAAAGIYGSGPGVFGPYGSAAAAAGPGSGIYGIGPYGPGASGPGIYGPGVFGPSASAAAAAGSGVFGPGIYGPYASAAAAAGIYGSGPGVFGPYGPGISGSGVFGPGVFGPYASAAAAAGPGVFGPYGPGSSAAAAAGIYGYGPGVFGPYGPGASGINGPGSGIYGPGVFGPGISAAAAAGPGVFGPYGPGASAAAAAGIYGPGVFGPGIYGPGSSGPGVFGPYGPGSSAAAAAGIYGPGVFGPYGPGISAAAAAGIYVFGPGVFGPYGPGASGPGVFGPYGPGASAAAAAGPGIYGPGVFGPSASAAAAAGIYGSGPGIYGPYGPGISGPGSGVFGIGPYGPGASAAAAAGIYGPGVFGPYGPGISAAAAAGPGSGIYGPGASGINGPGSGIYGPGVFGPGISAAAAAGIYVFGPGVFGPYGPGASAAAAAGIYGSGPGVFGPYGPGISGSGVFGPGVFGPYASAAAAAGPGSGVFGPGAS

SEQ ID NO: 33 - protein, 601 aa, Artificial Sequence
  PRT888
MHHHHHHSSGSSGPGVLGPYGPGASAAAAAGQNGPGSGVLGPGQSGQYGPGVLGPGVLGPGSSAAAAAGPGQYGPGVLGPSASAAAAAGPGSGVLGPGASGQYGPGVLGPGVLGPGSSAAAAAGQYGSGPGVLGPYGSAAAAAGPGSGQYGQGPYGPGASGPGQYGPGVLGPSASAAAAAGSGVLGPGQYGPYASAAAAAGQYGSGPGVLGPYGPGQSGSGVLGPGVLGPYASAAAAAGPGVLGPYGPGSSAAAAAGQYGYGPGVLGPYGPGASGQNGPGSGQYGPGVLGPGQSAAAAAGPGVLGPYGPGASAAAAAGQYGPGVLGPGQYGPGSSGPGVLGPYGPGSSAAAAAGQYGPGVLGPYGPGQSAAAAAGQYVLGPGVLGPYGPGASGPGVLGPYGPGASAAAAAGPGQYGPGVLGPSASAAAAAGQYGSGPGQYGPYGPGQSGPGSGVLGQGPYGPGASAAAAAGQYGPGVLGPYGPGQSAAAAAGPGSGQYGPGASGQNGPGSGQYGPGVLGPGQSAAAAAGQYVLGPGVLGPYGPGASAAAAAGQYGSGPGVLGPYGPGQSGSGVLGPGVLGPYASAAAAAGPGSGVLGPGAS

SEQ ID NO: 34 - protein, 601 aa, Artificial Sequence
  PRT965
MHHHHHHSSGSSGPGTSGPYGPGASAAAAAGANGPGSGTSGPGASGAYGPGTSGPGTSGPGSSAAAAAGPGAYGPGTSGPSASAAAAAGPGSGTSGPGASGAYGPGTSGPGTSGPGSSAAAAAGAYGSGPGTSGPYGSAAAAAGPGSGAYGAGPYGPGASGPGAYGPGTSGPSASAAAAAGSGTSGPGAYGPYASAAAAAGAYGSGPGTSGPYGPGASGSGTSGPGTSGPYASAAAAAGPGTSGPYGPGSSAAAAAGAYGYGPGTSGPYGPGASGANGPGSGAYGPGTSGPGASAAAAAGPGTSGPYGPGASAAAAAGAYGPGTSGPGAYGPGSSGPGTSGPYGPGSSAAAAAGAYGPGTSGPYGPGASAAAAAGAYTSGPGTSGPYGPGASGPGTSGPYGPGASAAAAAGPGAYGPGTSGPSASAAAAAGAYGSGPGAYGPYGPGASGPGSGTSGAGPYGPGASAAAAAGAYGPGTSGPYGPGASAAAAAGPGSGAYGPGASGANGPGSGAYGPGTSGPGASAAAAAGAYTSGPGTSGPYGPGASAAAAAGAYGSGPGTSGPYGPGASGSGTSGPGTSGPYASAAAAAGPGSGTSGPGAS

SEQ ID NO: 35 - protein, 601 aa, Artificial Sequence
  PRT889
MHHHHHHSSGSSGPGVLGPYGPGASAAAAAGINGPGSGVLGPGISGIYGPGVLGPGVLGPGSSAAAAAGPGIYGPGVLGPSASAAAAAGPGSGVLGPGASGIYGPGVLGPGVLGPGSSAAAAAGIYGSGPGVLGPYGSAAAAAGPGSGIYGIGPYGPGASGPGIYGPGVLGPSASAAAAAGSGVLGPGIYGPYASAAAAAGIYGSGPGVLGPYGPGISGSGVLGPGVLGPYASAAAAAGPGVLGPYGPGSSAAAAAGIYGYGPGVLGPYGPGASGINGPGSGIYGPGVLGPGISAAAAAGPGVLGPYGPGASAAAAAGIYGPGVLGPGIYGPGSSGPGVLGPYGPGSSAAAAAGIYGPGVLGPYGPGISAAAAAGIYVLGPGVLGPYGPGASGPGVLGPYGPGASAAAAAGPGIYGPGVLGPSASAAAAAGIYGSGPGIYGPYGPGISGPGSGVLGIGPYGPGASAAAAAGIYGPGVLGPYGPGISAAAAAGPGSGIYGPGASGINGPGSGIYGPGVLGPGISAAAAAGIYVLGPGVLGPYGPGASAAAAAGIYGSGPGVLGPYGPGISGSGVLGPGVLGPYASAAAAAGPGSGVLGPGAS

SEQ ID NO: 36 - protein, 601 aa, Artificial Sequence
  PRT916
MHHHHHHSSGSSGPGVIGPYGPGASAAAAAGLNGPGSGVIGPGLSGLYGPGVIGPGVIGPGSSAAAAAGPGLYGPGVIGPSASAAAAAGPGSGVIGPGASGLYGPGVIGPGVIGPGSSAAAAAGLYGSGPGVIGPYGSAAAAAGPGSGLYGLGPYGPGASGPGLYGPGVIGPSASAAAAAGSGVIGPGLYGPYASAAAAAGLYGSGPGVIGPYGPGLSGSGVIGPGVIGPYASAAAAAGPGVIGPYGPGSSAAAAAGLYGYGPGVIGPYGPGASGLNGPGSGLYGPGVIGPGLSAAAAAGPGVIGPYGPGASAAAAAGLYGPGVIGPGLYGPGSSGPGVIGPYGPGSSAAAAAGLYGPGVIGPYGPGLSAAAAAGLYVIGPGVIGPYGPGASGPGVIGPYGPGASAAAAAGPGLYGPGVIGPSASAAAAAGLYGSGPGLYGPYGPGLSGPGSGVIGLGPYGPGASAAAAAGLYGPGVIGPYGPGLSAAAAAGPGSGLYGPGASGLNGPGSGLYGPGVIGPGLSAAAAAGLYVIGPGVIGPYGPGASAAAAAGLYGSGPGVIGPYGPGLSGSGVIGPGVIGPYASAAAAAGPGSGVIGPGAS

SEQ ID NO: 37 - protein, 601 aa, Artificial Sequence
  PRT918
MHHHHHHSSGSSGPGVFGPYGPGASAAAAAGINGPGSGVFGPGISGIYGPGVFGPGVFGPGSSAAAAAGPGIYGPGVFGPSASAAAAAGPGSGVFGPGASGIYGPGVFGPGVFGPGSSAAAAAGIYGSGPGVFGPYGSAAAAAGPGSGIYGIGPYGPGASGPGIYGPGVFGPSASAAAAAGSGVFGPGIYGPYASAAAAAGIYGSGPGVFGPYGPGISGSGVFGPGVFGPYASAAAAAGPGVFGPYGPGSSAAAAAGIYGYGPGVFGPYGPGASGINGPGSGIYGPGVFGPGISAAAAAGPGVFGPYGPGASAAAAAGIYGPGVFGPGIYGPGSSGPGVFGPYGPGSSAAAAAGIYGPGVFGPYGPGISAAAAAGIYVFGPGVFGPYGPGASGPGVFGPYGPGASAAAAAGPGIYGPGVFGPSASAAAAAGIYGSGPGIYGPYGPGISGPGSGVFGIGPYGPGASAAAAAGIYGPGVFGPYGPGISAAAAAGPGSGIYGPGASGINGPGSGIYGPGVFGPGISAAAAAGIYVFGPGVFGPYGPGASAAAAAGIYGSGPGVFGPYGPGISGSGVFGPGVFGPYASAAAAAGPGSGVFGPGAS

SEQ ID NO: 38 - protein, 576 aa, Artificial Sequence
  PRT699
MHHHHHHSSGSSGPGVLGPYGPGASAAAAAAAGSNGPGSGVLGPGQSGQYGPGVLGPGVLGPGSSAAAAAAAGPGQYGPGVLGPSASAAAAAAAGPGSGVLGPGASGQYGPGVLGPGVLGPGSSAAAAAAAGSYGSGPGVLGPYGSAAAAAAAGPGSGQYGQGPYGPGASGPGQYGPGVLGPSASAAAAAAAGSGVLGPGQYGPYASAAAAAAAGSYGSGPGVLGPYGPGQSGSGVLGPGVLGPYASAAAAAAAGPGVLGPYGPGSSAAAAAAAGSYGYGPGVLGPYGPGASGQNGPGSGQYGPGVLGPGPSAAAAAAAGPGVLGPYGPGASAAAAAAAGSYGPGVLGPGQYGPGSSGPGVLGPYGPGSSAAAAAAAGSYGPGVLGPYGPGPSAAAAAAAGSYVLGPGVLGPYGPGASGPGVLGPYGPGASAAAAAAAGPGQYGPGVLGPSASAAAAAAAGSYGSGPGQYGPYGPGQSGPGSGVLGQGPYGPGASAAAAAAAGSYGPGVLGPYGPGPSAAAAAAAGPGSGQYGPGASGQNGPGSGQYGPGVLGPGPSAAAAAAAGPGSGVLGPGAS

SEQ ID NO: 39 - protein, 576 aa, Artificial Sequence
  PRT698
MHHHHHHSSGSSGPGVLGPYGPGASAAAAAAAGSNGPGSGVLGPGISGIYGPGVLGPGVLGPGSSAAAAAAAGPGIYGPGVLGPSASAAAAAAAGPGSGVLGPGASGIYGPGVLGPGVLGPGSSAAAAAAAGSYGSGPGVLGPYGSAAAAAAAGPGSGIYGIGPYGPGASGPGIYGPGVLGPSASAAAAAAAGSGVLGPGIYGPYASAAAAAAAGSYGSGPGVLGPYGPGISGSGVLGPGVLGPYASAAAAAAAGPGVLGPYGPGSSAAAAAAAGSYGYGPGVLGPYGPGASGINGPGSGIYGPGVLGPGPSAAAAAAAGPGVLGPYGPGASAAAAAAAGSYGPGVLGPGIYGPGSSGPGVLGPYGPGSSAAAAAAAGSYGPGVLGPYGPGPSAAAAAAAGSYVLGPGVLGPYGPGASGPGVLGPYGPGASAAAAAAAGPGIYGPGVLGPSASAAAAAAAGSYGSGPGIYGPYGPGISGPGSGVLGIGPYGPGASAAAAAAAGSYGPGVLGPYGPGPSAAAAAAAGPGSGIYGPGASGINGPGSGIYGPGVLGPGPSAAAAAAAGPGSGVLGPGAS

SEQ ID NO: 40 - protein, 1190 aa, Artificial Sequence
  PRT966
MHHHHHHSSGSSGPGVFGPYGPGASAAAAAGINGPGSGVFGPGISGIYGPGVFGPGVFGPGSSAAAAAGPGIYGPGVFGPSASAAAAAGPGSGVFGPGASGIYGPGVFGPGVFGPGSSAAAAAGIYGSGPGVFGPYGSAAAAAGPGSGIYGIGPYGPGASGPGIYGPGVFGPSASAAAAAGSGVFGPGIYGPYASAAAAAGIYGSGPGVFGPYGPGISGSGVFGPGVFGPYASAAAAAGPGVFGPYGPGSSAAAAAGIYGYGPGVFGPYGPGASGINGPGSGIYGPGVFGPGISAAAAAGPGVFGPYGPGASAAAAAGIYGPGVFGPGIYGPGSSGPGVFGPYGPGSSAAAAAGIYGPGVFGPYGPGISAAAAAGIYVFGPGVFGPYGPGASGPGVFGPYGPGASAAAAAGPGIYGPGVFGPSASAAAAAGIYGSGPGIYGPYGPGISGPGSGVFGIGPYGPGASAAAAAGIYGPGVFGPYGPGISAAAAAGPGSGIYGPGASGINGPGSGIYGPGVFGPGISAAAAAGIYVFGPGVFGPYGPGASAAAAAGIYGSGPGVFGPYGPGISGSGVFGPGVFGPYASAAAAAGPGSGVFGPGASGPGVFGPYGPGASAAAAAGINGPGSGVFGPGISGIYGPGVFGPGVFGPGSSAAAAAGPGIYGPGVFGPSASAAAAAGPGSGVFGPGASGIYGPGVFGPGVFGPGSSAAAAAGIYGSGPGVFGPYGSAAAAAGPGSGIYGIGPYGPGASGPGIYGPGVFGPSASAAAAAGSGVFGPGIYGPYASAAAAAGIYGSGPGVFGPYGPGISGSGVFGPGVFGPYASAAAAAGPGVFGPYGPGSSAAAAAGIYGYGPGVFGPYGPGASGINGPGSGIYGPGVFGPGISAAAAAGPGVFGPYGPGASAAAAAGIYGPGVFGPGIYGPGSSGPGVFGPYGPGSSAAAAAGIYGPGVFGPYGPGISAAAAAGIYVFGPGVFGPYGPGASGPGVFGPYGPGASAAAAAGPGIYGPGVFGPSASAAAAAGIYGSGPGIYGPYGPGISGPGSGVFGIGPYGPGASAAAAAGIYGPGVFGPYGPGISAAAAAGPGSGIYGPGASGINGPGSGIYGPGVFGPGISAAAAAGIYVFGPGVFGPYGPGASAAAAAGIYGSGPGVFGPYGPGISGSGVFGPGVFGPYASAAAAAGPGSGVFGPGAS

SEQ ID NO: 41 - protein, 590 aa, Artificial Sequence
  Met-PRT917
MGPGLIGPYGPGASAAAAAGVNGPGSGLIGPGVSGVYGPGLIGPGLIGPGSSAAAAAGPGVYGPGLIGPSASAAAAAGPGSGLIGPGASGVYGPGLIGPGLIGPGSSAAAAAGVYGSGPGLIGPYGSAAAAAGPGSGVYGVGPYGPGASGPGVYGPGLIGPSASAAAAAGSGLIGPGVYGPYASAAAAAGVYGSGPGLIGPYGPGVSGSGLIGPGLIGPYASAAAAAGPGLIGPYGPGSSAAAAAGVYGYGPGLIGPYGPGASGVNGPGSGVYGPGLIGPGVSAAAAAGPGLIGPYGPGASAAAAAGVYGPGLIGPGVYGPGSSGPGLIGPYGPGSSAAAAAGVYGPGLIGPYGPGVSAAAAAGVYLIGPGLIGPYGPGASGPGLIGPYGPGASAAAAAGPGVYGPGLIGPSASAAAAAGVYGSGPGVYGPYGPGVSGPGSGLIGVGPYGPGASAAAAAGVYGPGLIGPYGPGVSAAAAAGPGSGVYGPGASGVNGPGSGVYGPGLIGPGVSAAAAAGVYLIGPGLIGPYGPGASAAAAAGVYGSGPGLIGPYGPGVSGSGLIGPGLIGPYASAAAAAGPGSGLIGPGAS

SEQ ID NO: 42 - protein, 587 aa, Artificial Sequence
  Met-PRT1028
MGPGIFGPYGPGASAAAAAGTGPGSGIFGPGTSGTYGPGIFGPGIFGPGSSAAAAAGPGTYGPGIFGPSASAAAAAGPGSGIFGPGASGTYGPGIFGPGIFGPGSSAAAAAGTYGSGPGIFGPYGSAAAAAGPGSGTYGTGPYGPGASGPGTYGPGIFGPSASAAAAAGSGIFGPGTYGPYASAAAAAGTYGSGPGIFGPYGPGTSGSGIFGPGIFGPYASAAAAAGPGIFGPYGPGSSAAAAAGTYGYGPGIFGPYGPGASGTGPGSGTYGPGIFGPGTSAAAAAGPGIFGPYGPGASAAAAAGTYGPGIFGPGTYGPGSSGPGIFGPYGPGSSAAAAAGTYGPGIFGPYGPGTSAAAAAGTYIFGPGIFGPYGPGASGPGIFGPYGPGASAAAAAGPGTYGPGIFGPSASAAAAAGTYGSGPGTYGPYGPGTSGPGSGIFGTGPYGPGASAAAAAGTYGPGIFGPYGPGTSAAAAAGPGSGTYGPGASGTGPGSGTYGPGIFGPGTSAAAAAGTYIFGPGIFGPYGPGASAAAAAGTYGSGPGIFGPYGPGTSGSGIFGPGIFGPYASAAAAAGPGSGIFGPGAS

SEQ ID NO: 43 - protein, 601 aa, Artificial Sequence
  PRT917
MHHHHHHSSGSSGPGLIGPYGPGASAAAAAGVNGPGSGLIGPGVSGVYGPGLIGPGLIGPGSSAAAAAGPGVYGPGLIGPSASAAAAAGPGSGLIGPGASGVYGPGLIGPGLIGPGSSAAAAAGVYGSGPGLIGPYGSAAAAAGPGSGVYGVGPYGPGASGPGVYGPGLIGPSASAAAAAGSGLIGPGVYGPYASAAAAAGVYGSGPGLIGPYGPGVSGSGLIGPGLIGPYASAAAAAGPGLIGPYGPGSSAAAAAGVYGYGPGLIGPYGPGASGVNGPGSGVYGPGLIGPGVSAAAAAGPGLIGPYGPGASAAAAAGVYGPGLIGPGVYGPGSSGPGLIGPYGPGSSAAAAAGVYGPGLIGPYGPGVSAAAAAGVYLIGPGLIGPYGPGASGPGLIGPYGPGASAAAAAGPGVYGPGLIGPSASAAAAAGVYGSGPGVYGPYGPGVSGPGSGLIGVGPYGPGASAAAAAGVYGPGLIGPYGPGVSAAAAAGPGSGVYGPGASGVNGPGSGVYGPGLIGPGVSAAAAAGVYLIGPGLIGPYGPGASAAAAAGVYGSGPGLIGPYGPGVSGSGLIGPGLIGPYASAAAAAGPGSGLIGPGAS

SEQ ID NO: 44 - protein, 598 aa, Artificial Sequence
  PRT1028
MHHHHHHSSGSSGPGIFGPYGPGASAAAAAGTGPGSGIFGPGTSGTYGPGIFGPGIFGPGSSAAAAAGPGTYGPGIFGPSASAAAAAGPGSGIFGPGASGTYGPGIFGPGIFGPGSSAAAAAGTYGSGPGIFGPYGSAAAAAGPGSGTYGTGPYGPGASGPGTYGPGIFGPSASAAAAAGSGIFGPGTYGPYASAAAAAGTYGSGPGIFGPYGPGTSGSGIFGPGIFGPYASAAAAAGPGIFGPYGPGSSAAAAAGTYGYGPGIFGPYGPGASGTGPGSGTYGPGIFGPGTSAAAAAGPGIFGPYGPGASAAAAAGTYGPGIFGPGTYGPGSSGPGIFGPYGPGSSAAAAAGTYGPGIFGPYGPGTSAAAAAGTYIFGPGIFGPYGPGASGPGIFGPYGPGASAAAAAGPGTYGPGIFGPSASAAAAAGTYGSGPGTYGPYGPGTSGPGSGIFGTGPYGPGASAAAAAGTYGPGIFGPYGPGTSAAAAAGPGSGTYGPGASGTGPGSGTYGPGIFGPGTSAAAAAGTYIFGPGIFGPYGPGASAAAAAGTYGSGPGIFGPYGPGTSGSGIFGPGIFGPYASAAAAAGPGSGIFGPGAS

(19) United States
(12) Patent Application Publication
(10) Pub. No.: US 2022/0324923 A1
(43) Pub. Date: Oct. 13, 2022

(54) Method for Manufacturing Protein Molded Body

(71) Applicant: Spiber Inc., Tsuruoka-shi, Yamagata (JP)

(72) Inventors: Kentaro Takahashi, Tsuruoka-shi, Yamagata (JP); Atsushi Ikeda, Tsuruoka-shi, Yamagata (JP); Hideki Kagata, Tsuruoka-shi, Yamagata (JP); Yunosuke Abe, Tsuruoka-shi, Yamagata (JP); Kazuki Sakata, Tsuruoka-shi, Yamagata (JP)

(73) Assignee: Spiber Inc., Tsuruoka-shi, Yamagata (JP)

(21) Appl. No.: 17/764,669

(22) PCT Filed: Sep. 28, 2020
(86) PCT No.: PCT/JP2020/036613

§ 371 Date: Mar. 29, 2022

(30) Foreign Application Priority Data
Sep. 30, 2019 (JP) 2019-179958

(51) Int. Cl. C07K 14/435; D01F 4/02

(52) U.S. Cl. CPC C07K 14/43518; D01F 4/02

(57) ABSTRACT

An object of the present invention is to provide a method for advantageously producing a protein molded article which can solve a problem caused by esterification of a hydroxyl group contained in a protein while maintaining a sufficient strength. A method for producing a protein molded article according to the present invention includes a step of bringing a raw material molded article containing a protein in which a hydroxyl group is esterified into contact with an acidic or basic medium in a state of applying a tensile force, thus hydrolyzing an ester group.

### TECHNICAL FIELD

The present invention relates to a method for producing a protein molded article.

### BACKGROUND ART

Since a protein fiber such as silk has high biodegradability unlike a petroleum-derived synthetic fiber, with the recent increase in environmental problems, the protein fiber is expected to be used in various applications as an alternative for the synthetic fiber. As a method for producing a protein fiber, a method of spinning using an acid such as formic acid is widely known. For example, Patent Literature 1 discloses a method of treating a biological sample containing a structural polypeptide with an acid.

### CITATION LIST

### Patent Literature

### Patent Literature 1: JP 2004-503204 A

### SUMMARY OF INVENTION

### Technical Problem

The present inventors found that, in a protein fiber produced using a dope solution (spinning raw material solution) in which a carboxylic acid such as formic acid is used as a solvent, an ester group is formed by a dehydration condensation reaction between a hydroxyl group in a protein and the carboxylic acid during spinning. The present inventors further found that, in the protein fiber obtained as described above, hydrolysis of the ester group added to the protein proceeds with a trace amount of the carboxylic acid, such as formic acid, remaining on a surface or inside of the protein as a catalyst, a carboxylic acid may be thus released, and the released carboxylic acid causes a foul odor or the like. According to a study by the present inventors on the problem, it was found that, when a protein in which a hydroxyl group is esterified is brought into contact with an acidic or basic medium, and the ester group is thus hydrolyzed, the ester group causing the foul odor or the like can be removed or reduced.

Meanwhile, a protein molded article such as a protein fiber is expected to be used in various applications as described above, and therefore, it is desirable that the protein molded article has a sufficient strength in accordance with the application. However, it was found that a sufficient strength is not obtained by merely bringing the protein in which the hydroxyl group is esterified into contact with the acidic or basic medium to hydrolyze the ester group. The present invention is provided to solve such problems newly found by the present inventors.

That is, an object of the present invention is to provide a method for advantageously producing a protein molded article which can solve the problems caused by the esterification of a hydroxyl group contained in a protein while maintaining a sufficient strength. Another object of the present invention is to provide a method for processing a protein molded article which can solve the problems caused by the esterification of a hydroxyl group contained in a protein while maintaining a sufficient strength.

### Solution to Problem

For example, the present invention relates to each of the following inventions.

[1]

A method for producing a protein molded article including a step of bringing a raw material molded article containing a protein in which a hydroxyl group is esterified into contact with an acidic or basic medium in a state of applying a tensile force, thus hydrolyzing an ester group.

[2]

The method for producing a protein molded article according to [1], in which the medium is an aqueous solution.

[3]

The method for producing a protein molded article according to [2], in which the aqueous solution is an alkaline aqueous solution with a pH lower than 12.

[4]

The method for producing a protein molded article according to [2] or [3], in which the amount of the tensile force is an amount in which the raw material molded article does not shrink by the contact with the aqueous solution.

[5]

The method for producing a protein molded article according to any one of [1] to [4], in which the protein is a structural protein.

[6]

The method for producing a protein molded article according to [5], in which the structural protein is fibroin.

[7]

The method for producing a protein molded article according to [6], in which the fibroin is spider silk fibroin.

[8]

The method for producing a protein molded article according to any one of [1] to [7], in which the protein in which the hydroxyl group is esterified contains a formic acid ester.

[9]

The method for producing a protein molded article according to any one of [1] to [8], in which the raw material molded article is at least one selected from the group consisting of a fiber, a heat compression molded article, a film, a porous body, a gel, and a resin.

[10]

A method for processing a protein molded article including a step of bringing a protein molded article containing a protein in which a hydroxyl group is esterified into contact with an acidic or basic medium in a state of applying a tensile force, thus hydrolyzing an ester group.

### Advantageous Effects of Invention

According to the present invention, a method for advantageously producing a protein molded article is provided, which can solve the problems caused by esterification of a hydroxyl group contained in a protein while maintaining a sufficient strength. Furthermore, according to the present invention, a method for processing a protein molded article is provided, which can solve the problems caused by the esterification of a hydroxyl group contained in a protein while maintaining a sufficient strength.

### BRIEF DESCRIPTION OF DRAWINGS

FIG. 1 is a schematic view illustrating an example of a domain sequence of modified fibroin.

FIG. 2 is a schematic view illustrating an example of a domain sequence of modified fibroin.

FIG. 3 is a schematic view illustrating an example of a domain sequence of modified fibroin.

FIG. 4 is an FT-IR spectrum diagram of a protein fiber immediately after a hydrolysis treatment of an ester group (1,730 cm⁻¹: peak based on C═O of ester).

FIG. 5 is a diagram showing a stress (vertical axis)-degree of elongation (horizontal axis) curve in evaluation of a degree of elongation.

### DESCRIPTION OF EMBODIMENTS

Hereinafter, embodiments of the present invention will be described in detail. However, the present invention is not limited to the following embodiments.

[Method for Producing Protein Molded Article]

A method for producing a protein molded article according to the present embodiment includes a step of bringing a raw material molded article containing a protein in which a hydroxyl group is esterified into contact with an acidic or basic medium in a state of applying a tensile force, thus hydrolyzing an ester group.

In the present embodiment, the raw material molded article is at least one selected from the group consisting of a fiber, a heat compression molded article, a film, a porous body, a gel, and a resin.

In the present embodiment, the raw material molded article contains the protein in which a hydroxyl group is esterified. As used herein, the “protein in which a hydroxyl group is esterified” refers to a protein containing an ester group formed by formation of an ester bond between the hydroxyl group of the protein and a carboxylic acid. The protein in which a hydroxyl group is esterified may contain a formic acid ester, an acetic acid ester, a propionic acid ester, or the like, and the protein preferably contains a formic acid ester.

(Protein)

The protein according to the present embodiment (hereinafter, may be referred to as a “target protein”) may be, for example, a structural protein. The structural protein refers to a protein forming a biological structure or a protein derived therefrom. That is, the structural protein may be a naturally derived structural protein or a modified protein obtained by modifying a part of an amino acid sequence of a naturally derived structural protein (for example, 10% or less of the amino acid sequence) depending on the amino acid sequence.

Examples of the structural protein can include fibroin, collagen, resilin, elastin, keratin, and a protein derived from these proteins. The fibroin may be, for example, one or more selected from the group consisting of silk fibroin, spider silk fibroin, and hornet silk fibroin. The structural protein may be silk fibroin, spider silk fibroin, or a combination thereof.

The fibroin includes naturally derived fibroin and modified fibroin. As used herein, the “naturally derived fibroin” refers to fibroin having an amino acid sequence identical to that of naturally derived fibroin, and the “modified fibroin” refers to fibroin having an amino acid sequence different from that of the naturally derived fibroin.

The fibroin is preferably spider silk fibroin. The spider silk fibroin includes natural spider silk fibroin and modified fibroin derived from the natural spider silk fibroin. Examples of the natural spider silk fibroin include spider silk proteins produced by spiders.

Examples of the fibroin include a protein containing a domain sequence represented by Formula 1: [(A)ₙ motif-REP1]ₘ or Formula 2: [(A)/ₙ motif-REP1]ₘ−(A)ₙ motif. An amino acid sequence (N-terminal sequence or C-terminal sequence) may be further added to any one or both of the N-terminal side and the C-terminal side of the domain sequence of the fibroin. The N-terminal sequence and the C-terminal sequence are typically regions not containing repeats of amino acid motifs that are characteristic of fibroin and consist of about 100 residues of amino acids, but are not limited thereto.

As used herein, the “domain sequence” is an amino acid sequence giving rise to a crystalline region (typically corresponds to the (A)ₙ motif in the amino acid sequence) and a non-crystalline region (typically corresponds to REP in the amino acid sequence) characteristic of fibroin and refers to an amino acid sequence represented by Formula 1: [(A)ₙ motif-REP]ₘ or Formula 2: [(A)ₙ motif-REP]ₘ−(A)ₙ motif. Here, the (A)ₙ motif represents an amino acid sequence mainly consisting of alanine residues, and the number of amino acid residues therein is 2 to 27. The number of the amino acid residues in the (A)ₙ motif may be an integer of 2 to 20, 4 to 27, 4 to 20, 8 to 20, 10 to 20, 4 to 16, 8 to 16, or 10 to 16. In addition, a ratio of the number of alanine residues to the total number of the amino acid residues in the (A)ₙ motif may be 40% or higher, or may also be 60% or higher, 70% or higher, 80% or higher, 83% or higher, 85% or higher, 86% or higher, 90% or higher, 95% or higher, or 100% (meaning that the (A)ₙ motif only consists of alanine residues). Among the multiple (A)ₙ motifs in the domain sequence, at least 7 (A)ₙ motifs may only consist of alanine residues. REP represents an amino acid sequence consisting of 2 to 200 amino acid residues. REP may also be an amino acid sequence consisting of 10 to 200 amino acid residues. m represents an integer of 2 to 300, and may be an integer of 10 to 300. The multiple (A)ₙ motifs may be identical amino acid sequences or different amino acid sequences. The multiple REP's may be identical amino acid sequences or different amino acid sequences.

The modified fibroin can be obtained by, for example, performing amino acid sequence modification corresponding to a substitution, a deletion, an insertion, and/or an addition of one or a plurality of amino acid residues with respect to, for example, a cloned gene sequence for the naturally derived fibroin. The substitution, the deletion, the insertion, and/or the addition of an amino acid residue can be performed by a method known to those skilled in the art, such as a site-directed mutagenesis method. Specifically, the substitution, the deletion, the insertion, and/or the addition of an amino acid residue can be performed according to a method described in a literature such as Nucleic Acid Res. 10, 6487 (1982) and Methods in Enzymology, 100, 448 (1983).

The naturally derived fibroin is a protein containing a domain sequence represented by Formula 1: [(A)/ₙ motif-REP]ₘ or Formula 2: [(A)/ₙ motif-REP]ₘ−(A)ₙ motif, and specific examples thereof include fibroin produced by insects or spiders.

Examples of the fibroin produced by insects include silk proteins produced by silkworms such as Bombyx mori, Bombyx mandarina, Antheraea yamamai, Anteraea pernyi, Eriogyna pyretorum, Pilosamia Cynthia ricini, Samia cynthia, Caligura japonica, Antheraea mylitta, and Antheraea assama and a hornet silk protein secreted by larvae of Vespa simillima xanthoptera.

More specific examples of the fibroin produced by insects include the silkworm fibroin L chain (GenBank Accession Nos. M76430 (base sequence) and AAA27840.1 (amino acid sequence)).

Examples of the fibroin produced by arachnids include spider silk proteins produced by spiders belonging to the genus Araneus, such as Araneus ventricosus, Araneus diadematus, Araneus pinguis, Araneus pentagrammicus, and Araneus nojimai, spiders belonging to the genus Neoscona, such as Neoscona scylla, Neoscona nautica, Neoscona adianta, and Neoscona scylloides, spiders belonging to the genus Pronus, such as Pronous minutus, spiders belonging to the genus Cyrtarachne, such as Cyrtarachne bufo and Cyrtarachne inaequalis, spiders belonging to the genus Gasteracantha, such as Gasteracantha kuhlii and Gasteracantha mammosa, spiders belonging to the genus Ordgarius, such as Ordgarius hobsoni and Ordgarius sexspinosus, spiders belonging to the genus Argiope, such as Argiope amoena, Argiope minuta, and Argiope bruennichi, spiders belonging to the genus Arachnura, such as Arachnura logio, spiders belonging to the genus Acusilas, such as Acusilas coccineus, spiders belonging to the genus Cytophora, such as Cyrtophora moluccensis, Cyrtophora exanthematica, and Cyrtophora unicolor, spiders belonging to the genus Poltys, such as Poltys illepidus, spiders belonging to the genus Cyclosa, such as Cyclosa octotuberculata, Cyclosa sedeculata, Cyclosa vallata, and Cyclosa atrata, and spiders belonging to the genus Chorizopes, such as Chorizopes nipponicus, and spider silk proteins produced by spiders belonging to the family Tetragnathidae, such as spiders belonging to the genus Tetragnatha, such as Tetragnatha praedonia, Tetragnatha maxillosa, Tetragnatha extensa, and Tetragnatha squamata, spiders belonging to the genus Leucauge, such as Leucauge magnifica, Leucauge blanda, and Leucauge subblanda, spiders belonging to the genus Nephila, such as Nephila clavata and Nephila pilipes, spiders belonging to the genus Menosira, such as Menosira ornata, spiders belonging to the genus Dyschiriognatha, such as Dyschiriognatha tenera, spiders belonging to the genus Latrodectus, such as Latrodectus mactans, Latrodectus hasseltii, Latrodectus geometricus, and Latrodectus tredecimguttatus, and spiders belonging to the genus Euprosthenops. Examples of the spider silk proteins include dragline silk proteins such as MaSps (MaSp1 and MaSp2) and ADFs (ADF3 and ADF4), MiSps (MiSp1 and MiSp2), and the like.

More specific examples of the spider silk proteins produced by spiders include fibroin-3 (adf-3) [derived from Araneus diadematus] (GenBank accession numbers AAC47010 (amino acid sequence) and U47855 (base sequence)), fibroin-4 (adf-4) [derived from Araneus diadematus] (GenBank accession numbers AAC47011 (amino acid sequence) and U47856 (base sequence)), dragline silk protein spidroin 1 [derived from Nephila clavipes] (GenBank accession numbers AAC04504 (amino acid sequence) and U37520 (base sequence)), major ampullate spidroin 1 [derived from Latrodectus hesperus] (GenBank accession numbers ABR68856 (amino acid sequence) and EF595246 (base sequence)), dragline silk protein spidroin 2 [derived from Nephila clavata] (GenBank accession numbers AAL32472 (amino acid sequence) and AF441245 (base sequence)), major ampullate spidroin 1 [derived from Euprosthenops australis] (GenBank accession numbers CAJ00428 (amino acid sequence) and AJ973155 (base sequence)), and major ampullate spidroin 2 [Euprosthenops australis] (GenBank accession numbers CAM32249.1 (amino acid sequence) and AM490169 (base sequence)), minor ampullate silk protein 1 [Nephila clavipes] (GenBank accession number AAC14589.1 (amino acid sequence)), minor ampullate silk protein 2 [Nephila clavipes] (GenBank accession number AAC14591.1 (amino acid sequence)), minor ampullate spidroin-like protein [Nephilengys cruentata] (GenBank accession number ABR37278.1 (amino acid sequence), and the like.

More specific examples of the naturally derived fibroin can further include fibroin of which the sequence information is registered in NCBI GenBank. For example, the fibroin can be verified by extracting, from sequences containing INV as DIVISION, which is one of the sequence information registered in NCBI GenBank, a sequence having a keyword such as spidroin, ampullate, fibroin, “silk and polypeptide”, or “silk and protein” described under DEFINITION and a sequence having a specific character string of product described under CDS and a specific character string of TISSUE TYPE described under SOURCE.

The modified fibroin may be modified silk fibroin (fibroin obtained by modifying an amino acid sequence of a silk protein produced by silkworms), or may be modified spider silk fibroin (fibroin obtained by modifying an amino acid sequence of a spider silk protein produced by spiders). As the modified fibroin, the modified spider silk fibroin is preferred.

Specific examples of the modified fibroin include modified fibroin derived from a spigot dragline silk protein produced in a major ampullate gland of a spider (first modified fibroin), modified fibroin having a domain sequence in which a content of glycine residues is reduced (second modified fibroin), modified fibroin having a domain sequence in which a content of the (A)ₙ motifs is reduced (third modified fibroin), modified fibroin in which the contents of glycine residues and the (A)ₙ motifs are reduced (fourth modified fibroin), modified fibroin having a domain sequence containing a region in which a hydropathy index is locally high (fifth modified fibroin), and modified fibroin having a domain sequence in which a content of glutamine residues is reduced (sixth modified fibroin).

Examples of the first modified fibroin include a protein containing a domain sequence represented by Formula 1: [(A)ₙ motif-REP]ₘ. The number of amino acid residues in the (A)ₙ motif in the first modified fibroin is preferably an integer of 3 to 20, more preferably an integer of 4 to 20, even more preferably an integer of 8 to 20, still more preferably an integer of 10 to 20, still even more preferably an integer of 4 to 16, particularly preferably an integer of 8 to 16, and most preferably an integer of 10 to 16. The number of amino acid residues constituting REP in Formula 1 in the first modified fibroin is preferably 10 to 200 residues, more preferably 10 to 150 residues, even more preferably 20 to 100 residues, and still more preferably 20 to 75 residues. A total number of a glycine residue, a serine residue, and an alanine residue contained in the amino acid sequence represented by Formula 1: [(A)ₙ motif-REP]ₘ in the first modified fibroin is preferably 40% or more, more preferably 60% or more, and even more preferably 70% or more, with respect to the total number of amino acid residues.

The first modified fibroin may be a polypeptide which contains a unit of an amino acid sequence represented by Formula 1: [(A)ₙ motif-REP]ₘ and of which the C-terminal sequence is an amino acid sequence set forth in any one of SEQ ID NOs: 1 to 3 or an amino acid sequence having an identity of 90% or higher with an amino acid sequence set forth in any one of SEQ ID NOs: 1 to 3.

The amino acid sequence set forth in SEQ ID NO: 1 is identical to an amino acid sequence consisting of 50 amino acid residues at the C-terminus of the amino acid sequence of ADF3 (GI: 1263287, NCBI), the amino acid sequence set forth in SEQ ID NO: 2 is identical to an amino acid sequence obtained by removing 20 amino acid residues from the C-terminus of the amino acid sequence set forth in SEQ ID NO: 1, and the amino acid sequence set forth in SEQ ID NO: 3 is identical to an amino acid sequence obtained by removing 29 residues from the C-terminus of the amino acid sequence set forth in SEQ ID NO: 1.

More specific examples of the first modified fibroin can include modified fibroin containing (1-i) an amino acid sequence set forth in SEQ ID NO: 4 (recombinant spider silk protein ADF3KaiLargeNRSH1) or (1-ii) an amino acid sequence having a sequence identity of 90% or higher with the amino acid sequence set forth in SEQ ID NO: 4. It is preferable that the sequence identity is 95% or higher.

The amino acid sequence set forth in SEQ ID NO: 4 is obtained by causing mutations so that, in an amino acid sequence of ADF3 to which an amino acid sequence (SEQ ID NO: 5) consisting of a start codon, a His10-tag, and an HRV3C protease (human rhinovirus 3C protease) recognition site is added at the N-terminus, the 1^{st} to 13^{th} repeat regions are increased to be nearly doubled, and the translation is terminated at the 1,154^{th} amino acid residue. The C-terminal amino acid sequence of the amino acid sequence set forth in SEQ ID NO: 4 is identical to the amino acid sequence set forth in SEQ ID NO: 3.

The modified fibroin of (1-i) may consist of the amino acid sequence set forth in SEQ ID NO: 4.

The domain sequence of the second modified fibroin has an amino acid sequence in which the content of glycine residues is reduced compared to the naturally derived fibroin. The second modified fibroin can be defined as fibroin having an amino acid sequence corresponding to an amino acid sequence in which at least one or a plurality of glycine residues in REP are substituted by other amino acid residues, compared to the naturally derived fibroin.

The domain sequence of the second modified fibroin may have an amino acid sequence corresponding to an amino acid sequence in which one glycine residue in at least one or a plurality of motif sequences is substituted by another amino acid residue, compared to the naturally derived fibroin, the motif sequence being at least one motif sequence selected from GGX and GPGXX (where G represents a glycine residue, P represents a proline residue, and X represents an amino acid residue other than glycine) in REP.

In the second modified fibroin, a ratio of the above-described motif sequence in which a glycine residue is substituted by another amino acid residue to the total motif sequences may be 10% or higher.

The second modified fibroin contains a domain sequence represented by Formula 1: [(A)ₙ motif-REP]ₘ, and in a case where a total number of amino acid residues in amino acid sequences consisting of XGX (where X represents an amino acid residue other than glycine) contained in all REP's in the domain sequence excluding a sequence from the (A)ₙ motif located at the most C-terminal side to the C-terminus of the domain sequence is denoted by z, and a total number of amino acid residues in the domain sequence excluding the sequence from the (A)ₙ motif located at the most C-terminal side to the C-terminus of the domain sequence is denoted by w, the second modified fibroin may have an amino acid sequence in which z/w is 30% or higher, 40% or higher, 50% or higher, or 50.9% or higher. The number of alanine residues with respect to the total number of amino acid residues in the (A)ₙ motif may be 83% or higher, and the number of alanine residues with respect to the total number of amino acid residues in the (A)ₙ motif is preferably 86% or higher, more preferably 90% or higher, even more preferably 95% or higher, and still more preferably 100% (meaning that the (A)ₙ motif only consists of alanine residues).

It is preferable that a content ratio of the amino acid sequence consisting of XGX in the second modified fibroin is increased by substituting one glycine residue in the GGX motif with another amino acid residue. A content ratio of the amino acid sequence consisting of GGX in the domain sequence of the second modified fibroin is preferably 30% or lower, more preferably 20% or lower, even more preferably 10% or lower, still more preferably 6% or lower, still even more preferably 4% or lower, and particularly preferably 2% or lower. The content ratio of the amino acid sequence consisting of GGX in the domain sequence can be calculated by the same method as the method for calculating the content ratio of the amino acid sequence consisting of XGX (z/w) below.

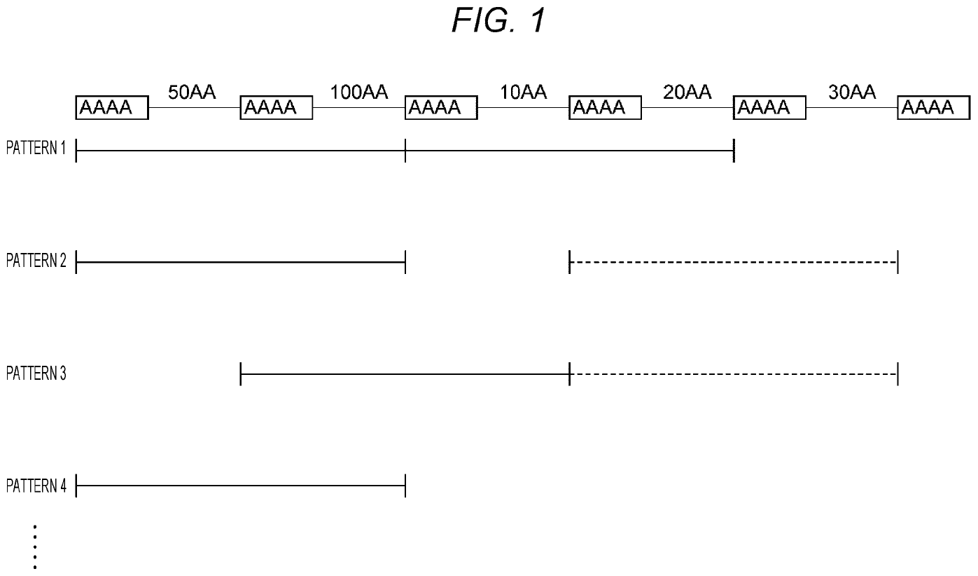

The method for calculating z/w will be described in further detail. First, the amino acid sequence consisting of XGX is extracted from all REP's contained in a domain sequence represented by Formula 1: [(A)ₙ motif-REP]ₘ in fibroin (modified fibroin or naturally derived fibroin) excluding a sequence from the (A)ₙ motif located at the most C-terminal side to the C-terminus of the domain sequence. A total number of amino acid residues constituting XGX is denoted by z. For example, in a case where 50 amino acid sequences consisting of XGX are extracted (without overlaps), z is 50×3=150. Furthermore, in a case where X belonging to two XGX sequences is present, as in the case of, for example, an amino acid sequence consisting of XGXGX (X in the center), z is calculated by deducting the overlapping amino acid residue (in the case of XGXGX, the number of amino acid residues is 5). w is a total number of amino acid residues in the domain sequence excluding the sequence from the (A)ₙ motif located at the most C-terminal side to the C-terminus of the domain sequence. For example, in a case of the domain sequence shown in FIG. 1, w is 4+50+4+100+4+10+4+20+4+30=230 (the (A)ₙ motif located at the most C-terminal side is excluded). Next, z/w (%) can be calculated by dividing z by w.

Here, z/w in the naturally derived fibroin will be described. First, fibroin of which the amino acid sequence information is registered in NCBI GenBank was verified using the method exemplified above, and as a result, 663 types of fibroin (among these, 415 types were fibroin derived from spiders) were extracted. Among all extracted fibroin, values of z/w were calculated using the calculation method described above from amino acid sequences of naturally derived fibroin which contained domain sequences represented by Formula 1: [(A)ₙ motif-REP]ₘ and in which the content ratios of the amino acid sequences consisting of GGX were 6% or lower. As a result, the values of z/w in the naturally derived fibroin are all smaller than 50.9% (the largest value is 50.86%).

z/w in the second modified fibroin is preferably 50.9% or higher, more preferably 56.1% or higher, even more preferably 58.7% or higher, still more preferably 70% or higher, and still even more preferably 80% or higher. The upper limit of z/w is not particularly limited, and may be, for example, 95% or lower.

The second modified fibroin can be obtained by, for example, performing modification so that at least a part of base sequences encoding glycine residues in a cloned gene sequence for the naturally derived fibroin are substituted so as to encode another amino acid residue. In this case, one glycine residue in the GGX motif and the GPGXX motif may be selected as the glycine residue to be modified, and the substitution may be performed so that z/w is 50.9% or higher. It is also possible to obtain the second modified fibroin by, for example, designing an amino acid sequence satisfying the above aspect from the amino acid sequence of the naturally derived fibroin and chemically synthesizing a nucleic acid encoding the designed amino acid sequence. In any case, in addition to the modification corresponding to a substitution of a glycine residue in REP in the amino acid sequence of the naturally derived fibroin with another amino acid residue, further amino acid sequence modification may be performed, which corresponds to a substitution, a deletion, an insertion, and/or an addition of one or a plurality of amino acid residues.

Another amino acid residue above is not particularly limited as long as it is an amino acid residue other than a glycine residue, and the amino acid residue is preferably a hydrophobic amino acid residue such as a valine (V) residue, a leucine (L) residue, an isoleucine (I) residue, a methionine (M) residue, a proline (P) residue, a phenylalanine (F) residue, and a tryptophan (W) residue, and a hydrophilic amino acid residue such as a glutamine (Q) residue, an asparagine (N) residue, a serine (S) residue, a lysine (K) residue, and a glutamic acid (E) residue, more preferably a valine (V) residue, a leucine (L) residue, an isoleucine (I) residue, a phenylalanine (F) residue, and a glutamine (Q) residue, and even more preferably a glutamine (Q) residue.

More specific examples of the second modified fibroin can include modified fibroin containing (2-i) an amino acid sequence set forth in SEQ ID NO: 6 (Met-PRT380), SEQ ID NO: 7 (Met-PRT410), SEQ ID NO: 8 (Met-PRT525), or SEQ ID NO: 9 (Met-PRT799) or (2-ii) an amino acid sequence having a sequence identity of 90% or higher with the amino acid sequence set forth in SEQ ID NO: 6, SEQ ID NO: 7, SEQ ID NO: 8, or SEQ ID NO: 9.

The modified fibroin of (2-i) will be described. The amino acid sequence set forth in SEQ ID NO: 6 is obtained by substituting all GGX's in REP in an amino acid sequence set forth in SEQ ID NO: 10 (Met-PRT313), which corresponds to the naturally derived fibroin, with GQX's. The amino acid sequence set forth in SEQ ID NO: 7 is obtained from the amino acid sequence set forth in SEQ ID NO: 6, by deleting every other two (A)ₙ motifs from the N-terminal side to the C-terminal side and inserting one [(A)ₙ motif-REP] before the C-terminal sequence. The amino acid sequence set forth in SEQ ID NO: 8 is obtained by inserting two alanine residues on the C-terminal side of each (A)ₙ motif in the amino acid sequence forth in SEQ ID NO: 7, substituting a part of glutamine (Q) residues with serine (S) residues, and deleting a part of amino acids on the C-terminal side so that the molecular weight thereof is about the same as the molecular weight of the amino acid sequence set forth in SEQ ID NO: 7. The amino acid sequence set forth in SEQ ID NO: 9 is obtained by adding a predetermined hinge sequence and His-tag sequence to the C-terminus of a sequence in which a region of 20 domain sequences (here, several amino acid residues on the C-terminal side of the region are substituted) existing in the amino acid sequence set forth in SEQ ID NO: 7 is repeated 4 times.

A value of z/w in the amino acid sequence set forth in SEQ ID NO: 10 (corresponding to naturally derived fibroin) is 46.8%. Values of z/w in the amino acid sequence set forth in SEQ ID NO: 6, the amino acid sequence set forth in SEQ ID NO: 7, the amino acid sequence set forth in SEQ ID NO: 8, and the amino acid sequence set forth in SEQ ID NO: 9 are 58.7%, 70.1%, 66.1%, and 70.0%, respectively. Furthermore, values of x/y in the amino acid sequences set forth in SEQ ID NO: 10, SEQ ID NO: 6, SEQ ID NO: 7, SEQ ID NO: 8, and SEQ ID NO: 9 at a Giza ratio (to be described later) of 1:1.8 to 11.3 are 15.0%, 15.0%, 93.4%, 92.7%, and 89.8%, respectively.

The modified fibroin of (2-i) may consist of the amino acid sequence set forth in SEQ ID NO: 6, SEQ ID NO: 7, SEQ ID NO: 8, or SEQ ID NO: 9.

The modified fibroin of (2-ii) contains an amino acid sequence having a sequence identity of 90% or higher with the amino acid sequence set forth in SEQ ID NO: 6, SEQ ID NO: 7, SEQ ID NO: 8, or SEQ ID NO: 9. The modified fibroin of (2-ii) is also a protein containing a domain sequence represented by Formula 1: [(A)ₙ motif-REP]ₘ. It is preferable that the sequence identity is 95% or higher.

The modified fibroin of (2-ii) has a sequence identity of 90% or higher with the amino acid sequence set forth in SEQ ID NO: 6, SEQ ID NO: 7, SEQ ID NO: 8, or SEQ ID NO: 9, and in a case where a total number of amino acid residues in amino acid sequences consisting of XGX (where X represents an amino acid residue other than glycine) which are contained in REP is denoted by z, and a total number of amino acid residues in REP's in the domain sequence is denoted by w, z/w is preferably 50.9% or higher.

The second modified fibroin may contain a tag sequence at one or both of the N-terminus and the C-terminus thereof. By containing a tag sequence, isolation, immobilization, detection, visualization, and the like of the modified fibroin become possible.

Examples of the tag sequence can include an affinity tag using specific affinity (binding properties or affinity) to another molecule. Specific examples of the affinity tag can include a histidine tag (His-tag). The His-tag is a short peptide in which about 4 to 10 histidine residues are lined up and can be used for isolating modified fibroin by chelating metal chromatography, since it has a property of specifically binding to metal ions such as nickel. Specific examples of the tag sequence include an amino acid sequence set forth in SEQ ID NO: 11 (an amino acid sequence containing a His-tag sequence and a hinge sequence).

Furthermore, tag sequences such as a glutathione S-transferase (GST) that specifically binds to glutathione and maltose-binding protein (MBP) that specifically binds to maltose can also be used.

In addition, an “epitope tag” using an antigen-antibody reaction can also be used. By adding a peptide exhibiting antigenicity (epitope) as a tag sequence, an antibody to the epitope can bind to the modified fibroin. Examples of the epitope tag can include an HA (a peptide sequence of influenza virus hemagglutinin) tag, a myc tag, a FLAG tag, and the like. The use of the epitope tag allows purification of the modified fibroin to be easily performed with high specificity.

In addition, a tag sequence that can be cleaved by a specific protease can also be used. By treating a protein adsorbed via the tag sequence with a protease, the modified fibroin from which the tag sequence is cleaved can be recovered.

More specific examples of the modified fibroin containing a tag sequence can include modified fibroin containing (2-iii) an amino acid sequence set forth in SEQ ID NO: 12 (PRT380), SEQ ID NO: 13 (PRT410), SEQ ID NO: 14 (PRT525), or SEQ ID NO: 15 (PRT799) or (2-iv) an amino acid sequence having a sequence identity of 90% or higher with the amino acid sequence set forth in SEQ ID NO: 12, SEQ ID NO: 13, SEQ ID NO: 14, or SEQ ID NO: 15.

The amino acid sequences set forth in SEQ ID NO: 16 (PRT313), SEQ ID NO: 12, SEQ ID NO: 13, SEQ ID NO: 14, and SEQ ID NO: 15 are obtained by adding the amino acid sequence set forth in SEQ ID NO: 11 (which contains a His-tag sequence and a hinge sequence) to the N-termini of the amino acid sequences set forth in SEQ ID NO: 10, SEQ ID NO: 6, SEQ ID NO: 7, SEQ ID NO: 8, and SEQ ID NO: 9, respectively.

The modified fibroin of (2-iii) may consist of the amino acid sequence set forth in SEQ ID NO: 12, SEQ ID NO: 13, SEQ ID NO: 14, or SEQ ID NO: 15.

The modified fibroin of (2-iv) contains an amino acid sequence having a sequence identity of 90% or higher with the amino acid sequence set forth in SEQ ID NO: 12, SEQ ID NO: 13, SEQ ID NO: 14, or SEQ ID NO: 15. The modified fibroin of (2-iv) is also a protein containing a domain sequence represented by Formula 1: [(A)ₙ motif-REP]ₘ. It is preferable that the sequence identity is 95% or higher.

The modified fibroin of (2-iv) has a sequence identity of 90% or higher with the amino acid sequence set forth in SEQ ID NO: 12, SEQ ID NO: 13, SEQ ID NO: 14, or SEQ ID NO: 15, and in a case where a total number of amino acid residues in amino acid sequences consisting of XGX (where X represents an amino acid residue other than glycine) which are contained in REP is denoted by z, and a total number of amino acid residues in REP's in the domain sequence is denoted by w, z/w is preferably 50.9% or higher.

The second modified fibroin may contain a secretory signal for releasing a protein produced in a recombinant protein production system to the outside of a host. A sequence of the secretory signal can be suitably set according to the type of the host.

The domain sequence of the third modified fibroin has an amino acid sequence in which the content of the (A)ₙ motifs is reduced compared to the naturally derived fibroin. The domain sequence of the third modified fibroin can be defined as a domain sequence having an amino acid sequence corresponding to an amino acid sequence in which at least one or a plurality of the (A)ₙ motifs are deleted, compared to the naturally derived fibroin.

The third modified fibroin may have an amino acid sequence corresponding to an amino acid sequence obtained by deleting 10% to 40% of the (A)ₙ motifs in the naturally derived fibroin.

The domain sequence of the third modified fibroin may have an amino acid sequence corresponding to an amino acid sequence in which at least one (A)ₙ motif in every one to three (A)ₙ motifs is deleted from the N-terminal side to the C-terminal side, compared to the naturally derived fibroin.

The domain sequence of the third modified fibroin may have an amino acid sequence corresponding to an amino acid sequence in which, at least, a deletion of two consecutive (A)ₙ motifs and a deletion of one (A)ₙ motif are repeated in this order from the N-terminal side to the C-terminal side, compared to the naturally derived fibroin.

The domain sequence of the third modified fibroin may have an amino acid sequence corresponding to an amino acid sequence in which at least every other two (A)ₙ motifs are deleted from the N-terminal side to the C-terminal side.

The third modified fibroin has a domain sequence represented by Formula 1: [(A)ₙ motif-REP]ₘ, and in a case of summing up the numbers of amino acid residues in two adjacent [(A)ₙ motif-REP] units in which, when the numbers of amino acid residues in REP's of two adjacent [(A)ₙ motif-REP] units are sequentially compared from the N-terminal side to the C-terminal side and the number of amino acid residues in REP having a smaller number of amino acid residues is set as 1, the ratio of the number of the amino acid residues in the other REP is 1.8 to 11.3, and denoting the maximum value of the sum by x and the total number of amino acid residues in the domain sequence by y, the third modified fibroin may have an amino acid sequence in which x/y is 20% or higher, 30% or higher, 40% or higher, or 50% or higher. The number of alanine residues with respect to the total number of amino acid residues in the (A)ₙ motif may be 83% or higher, and the number of alanine residues with respect to the total number of amino acid residues in the (A)ₙ motif is preferably 86% or higher, more preferably 90% or higher, even more preferably 95% or higher, and still more preferably 100% (meaning that the (A)ₙ motif only consists of alanine residues).

The method for calculating x/y will be described in further detail with reference to FIG. 1. FIG. 1 shows a domain sequence excluding the N-terminal sequence and the C-terminal sequence from the modified fibroin. The domain sequence has a sequence (A)ₙ motif-first REP (50 amino acid residues)−(A)ₙ motif-second REP (100 amino acid residues)-(A)ₙ motif-third REP (10 amino acid residues)−(A)ₙ motif-fourth REP (20 amino acid residues)−(A)ₙ motif-fifth REP (30 amino acid residues)−(A)ₙ motif from the N-terminal side (left side).

Two adjacent [(A)ₙ motif-REP] units are sequentially selected from the N-terminal side to the C-terminal side without overlaps. At this time, an unselected [(A)ₙ motif-REP] unit may exist. FIG. 1 shows a pattern 1 (comparison between the first REP and the second REP and comparison between the third REP and the fourth REP), a pattern 2 (comparison between the first REP and the second REP and comparison between the fourth REP and the fifth REP), a pattern 3 (comparison between the second REP and the third REP and comparison between the fourth REP and the fifth REP), and a pattern 4 (comparison between the first REP and the second REP). There are other selection methods besides this.

Next, in each pattern, the numbers of amino acid residues in the REP's of the selected two adjacent [(A)ₙ motif-REP] units are compared with each other. The comparison is performed by setting the smaller number of amino acid residues as 1 and determining the ratio of the number of amino acid residues in the other REP. For example, in the case of comparing the first REP (50 amino acid residues) and the second REP (100 amino acid residues), when the number of amino acid residues in the first REP which is smaller is set as 1, the ratio of the number of amino acid residues in the second REP is 100/50=2. In the same manner, in the case of comparing the fourth REP (20 amino acid residues) and the fifth REP (30 amino acid residues), when the number of amino acid residues in the fourth REP which is smaller is set as 1, the ratio of the number of amino acid residues in the fifth REP is 30/20=1.5.

In FIG. 1, a set of [(A)ₙ motif-REP] units in which, when the smaller number of amino acid residues is set as 1, the ratio of the number of amino acid residues in the other REP is 1.8 to 11.3 is indicated by a solid line. In the present specification, this ratio will be referred to as a Giza ratio. A set of [(A)ₙ motif-REP] units in which, when the smaller number of amino acid residues is set as 1, the ratio of the number of amino acid residues in the other REP is smaller than 1.8 or exceeds 11.3 is indicated by a dashed line.

In each pattern, all of the numbers of amino acid residues in the two adjacent [(A)ₙ motif-REP] units indicated by the solid lines (including not only the number of amino acid residues in REP but also the number of amino acid residues in the (A)ₙ motif) are summed up. The values of the sums are compared with each other, and the value of the sum of a pattern with the largest sum (maximum value of the sum) is denoted by x. In the example illustrated in FIG. 1, the value of the sum is maximum in the pattern 1.

Then, x/y (%) can be calculated by dividing x by the total number of amino acid residues y of the domain sequence.

x/y in the third modified fibroin is preferably 50% or higher, more preferably 60% or higher, even more preferably 65% or higher, still more preferably 70% or higher, still even more preferably 75% or higher, and particularly preferably 80% or higher. The upper limit of x/y is not particularly limited, and may be, for example, 100% or lower. In a case where the Giza ratio is 1:1.9 to 11.3, x/y is preferably 89.6% or higher, in a case where the Giza ratio is 1:1.8 to 3.4, x/y is preferably 77.1% or higher, in a case where the Giza ratio is 1:1.9 to 8.4, x/y is preferably 75.9% or higher, and in a case where the Giza ratio is 1:1.9 to 4.1, x/y is preferably 64.2% or higher.

In a case where the third modified fibroin is modified fibroin in which at least 7 of a plurality of (A)ₙ motifs present in the domain sequence only consist of alanine residues, x/y is preferably 46.4% or higher, more preferably 50% or higher, even more preferably 55% or higher, still more preferably 60% or higher, still even more preferably 70% or higher, and particularly preferably 80% or higher. The upper limit of x/y is not particularly limited and may be 100% or lower.

Here, x/y in the naturally derived fibroin will be described. First, fibroin of which the amino acid sequence information is registered in NCBI GenBank was verified using the method exemplified above, and as a result, 663 types of fibroin (among these, 415 types were fibroin derived from spiders) were extracted. Among all extracted fibroin, values of x/y were calculated using the calculation method described above from amino acid sequences of naturally derived fibroin consisting of domain sequences represented by Formula 1: [(A)ₙ motif-REP]ₘ. As a result, the values of x/y in the naturally derived fibroin are all smaller than 64.2% (the largest value is 64.14%).

The third modified fibroin can be obtained by, for example, deleting one or a plurality of sequences encoding the (A)ₙ motif from a cloned gene sequence for the naturally derived fibroin so that x/y is 64.2% or higher. It is also possible to obtain the third modified fibroin by, for example, designing an amino acid sequence corresponding to an amino acid sequence obtained by deleting one or a plurality of (A)ₙ motifs from the amino acid sequence of the naturally derived fibroin so that x/y is 64.2% or higher and chemically synthesizing a nucleic acid encoding the designed amino acid sequence. In any case, in addition to the modification corresponding to deletion of the (A)ₙ motif from the amino acid sequence of the naturally derived fibroin, further amino acid sequence modification may be performed, which corresponds to a substitution, a deletion, an insertion, and/or an addition of one or a plurality of amino acid residues.

More specific examples of the third modified fibroin can include modified fibroin containing (3-i) an amino acid sequence set forth in SEQ ID NO: 17 (Met-PRT399), SEQ ID NO: 7 (Met-PRT410), SEQ ID NO: 8 (Met-PRT525), or SEQ ID NO: 9 (Met-PRT799) or (3-ii) an amino acid sequence having a sequence identity of 90% or higher with the amino acid sequence set forth in SEQ ID NO: 17, SEQ ID NO: 7, SEQ ID NO: 8, or SEQ ID NO: 9.

The modified fibroin of (3-i) will be described. The amino acid sequence set forth in SEQ ID NO: 17 is obtained from the amino acid sequence set forth in SEQ ID NO: 10 (Met-PRT313) that corresponds to the naturally derived fibroin, by deleting every other two (A)ₙ motifs from the N-terminal side to the C-terminal side and inserting one [(A)ₙ motif-REP] before the C-terminal sequence. The amino acid sequence set forth in SEQ ID NO: 7, SEQ ID NO: 8, or SEQ ID NO: 9 is as described in the second modified fibroin.

A value of x/y in the amino acid sequence set forth in SEQ ID NO: 10 (corresponding to the naturally derived fibroin) at a Giza ratio of 1:1.8 to 11.3 is 15.0%. Values of x/y in the amino acid sequence set forth in SEQ ID NO: 17 and the amino acid sequence set forth in SEQ ID NO: 7 are both 93.4%. A value of x/y in the amino acid sequence set forth in SEQ ID NO: 8 is 92.7%. A value of x/y in the amino acid sequence set forth in SEQ ID NO: 9 is 89.8%. Values of z/w in the amino acid sequences set forth in SEQ ID NO: 10, SEQ ID NO: 17, SEQ ID NO: 7, SEQ ID NO: 8, and SEQ ID NO: 9 are 46.8%, 56.2%, 70.1%, 66.1%, and 70.0%, respectively.

The modified fibroin of (3-i) may consist of the amino acid sequence set forth in SEQ ID NO: 17, SEQ ID NO: 7, SEQ ID NO: 8, or SEQ ID NO: 9.

The modified fibroin of (3-ii) contains an amino acid sequence having a sequence identity of 90% or higher with the amino acid sequence set forth in SEQ ID NO: 17, SEQ ID NO: 7, SEQ ID NO: 8, or SEQ ID NO: 9. The modified fibroin of (3-ii) is also a protein containing a domain sequence represented by Formula 1: [(A)ₙ motif-REP]ₘ. It is preferable that the sequence identity is 95% or higher.

The modified fibroin of (3-ii) has a sequence identity of 90% or higher with the amino acid sequence set forth in SEQ ID NO: 17, SEQ ID NO: 7, SEQ ID NO: 8, or SEQ ID NO: 9, and in a case of summing up the numbers of amino acid residues in two adjacent [(A)ₙ motif-REP] units in which, when the numbers of amino acid residues in REP's of two adjacent [(A)ₙ motif-REP] units are sequentially compared from the N-terminal side to the C-terminal side and the number of amino acid residues in REP having a smaller number of amino acid residues is set as 1, the ratio of the number of the amino acid residues in the other REP is 1.8 to 11.3 (a Giza ratio is 1:1.8 to 11.3), and denoting the maximum value of the sum by x and the total number of amino acid residues in the domain sequence by y, x/y is preferably 64.2% or higher.

The third modified fibroin may contain the tag sequence described above at one or both of the N-terminus and the C-terminus thereof.

More specific examples of the modified fibroin containing a tag sequence can include modified fibroin containing (3-iii) an amino acid sequence set forth in SEQ ID NO: 18 (PRT399), SEQ ID NO: 13 (PRT410), SEQ ID NO: 14 (PRT525), or SEQ ID NO: 15 (PRT799) or (3-iv) an amino acid sequence having a sequence identity of 90% or higher with the amino acid sequence set forth in SEQ ID NO: 18, SEQ ID NO: 13, SEQ ID NO: 14, or SEQ ID NO: 15.

The amino acid sequences set forth in SEQ ID NO: 18, SEQ ID NO: 13, SEQ ID NO: 14, and SEQ ID NO: 15 are obtained by adding the amino acid sequence set forth in SEQ ID NO: 11 (which contains a His-tag sequence and a hinge sequence) to the N-termini of the amino acid sequences set forth in SEQ ID NO: 17, SEQ ID NO: 7, SEQ ID NO: 8, and SEQ ID NO: 9, respectively.

The modified fibroin of (3-iii) may consist of the amino acid sequence set forth in SEQ ID NO: 18, SEQ ID NO: 13, SEQ ID NO: 14, or SEQ ID NO: 15.

The modified fibroin of (3-iv) contains an amino acid sequence having a sequence identity of 90% or higher with the amino acid sequence set forth in SEQ ID NO: 18, SEQ ID NO: 13, SEQ ID NO: 14, or SEQ ID NO: 15. The modified fibroin of (3-iv) is also a protein containing a domain sequence represented by Formula 1: [(A)ₙ motif-REP]ₘ. It is preferable that the sequence identity is 95% or higher.

The modified fibroin of (3-iv) has a sequence identity of 90% or higher with the amino acid sequence set forth in SEQ ID NO: 18, SEQ ID NO: 13, SEQ ID NO: 14, or SEQ ID NO: 15, and in a case of summing up the numbers of amino acid residues in two adjacent [(A)ₙ motif-REP] units in which, when the numbers of amino acid residues in REP's of two adjacent [(A)ₙ motif-REP] units are sequentially compared from the N-terminal side to the C-terminal side and the number of amino acid residues in REP having a smaller number of amino acid residues is set as 1, the ratio of the number of the amino acid residues in the other REP is 1.8 to 11.3, and denoting the maximum value of the sum by x and the total number of amino acid residues in the domain sequence by y, x/y is preferably 64.2% or higher.

The third modified fibroin may contain a secretory signal for releasing a protein produced in a recombinant protein production system to the outside of a host. A sequence of the secretory signal can be suitably set according to the type of the host.

The domain sequence of the fourth modified fibroin has an amino acid sequence having a reduced content of glycine residues, as well as a reduced content of the (A)ₙ motifs, compared to the naturally derived fibroin. The domain sequence of the fourth modified fibroin can be defined as a domain sequence having an amino acid sequence corresponding to an amino acid sequence in which at least one or a plurality of the (A)ₙ motifs are deleted, and at least one or a plurality of glycine residues in REP are substituted by other amino acid residues, compared to the naturally derived fibroin. That is, the fourth modified fibroin is modified fibroin having characteristics of both the second modified fibroin and the third modified fibroin described above. Specific aspects thereof and the like are as in the descriptions for the second modified fibroin and the third modified fibroin.

More specific examples of the fourth modified fibroin can include modified fibroin containing (4-i) an amino acid sequence set forth in SEQ ID NO: 7 (Met-PRT410), SEQ ID NO: 8 (Met-PRT525), SEQ ID NO: 9 (Met-PRT799), SEQ ID NO: 13 (PRT410), SEQ ID NO: 14 (PRT525), or SEQ ID NO: 15 (PRT799) or (4-ii) an amino acid sequence having a sequence identity of 90% or higher with the amino acid sequence set forth in SEQ ID NO: 7, SEQ ID NO: 8, SEQ ID NO: 9, SEQ ID NO: 13, SEQ ID NO: 14, or SEQ ID NO: 15. Specific aspects of the modified fibroin containing the amino acid sequence set forth in SEQ ID NO: 7, SEQ ID NO: 8, SEQ ID NO: 9, SEQ ID NO: 13, SEQ ID NO: 14, or SEQ ID NO: 15 are as described above.

The domain sequence of the fifth modified fibroin may have an amino acid sequence containing a region in which a hydropathy index is locally high, which corresponds to an amino acid sequence in which one or a plurality of amino acid residues in REP are substituted by amino acid residues having a high hydropathy index, and/or an amino acid sequence in which one or a plurality of amino acid residues having a high hydropathy index are inserted into REP, compared to the naturally derived fibroin.

It is preferable that the region in which a hydropathy index is locally high consists of 2 to 4 consecutive amino acid residues.

The amino acid residue having a high hydropathy index described above is more preferably an amino acid residue selected from isoleucine (I), valine (V), leucine (L), phenylalanine (F), cysteine (C), methionine (M), and alanine (A).

In addition to the modification corresponding to a substitution of one or a plurality of amino acid residues in REP with amino acid residues having a high hydropathy index and/or an insertion of one or a plurality of amino acid residues having a high hydropathy index into REP, compared to the naturally derived fibroin, further amino acid sequence modification may be performed on the fifth modified fibroin, which corresponds to a substitution, a deletion, an insertion, and/or an addition of one or a plurality of amino acid residues, compared to the naturally derived fibroin.

The fifth modified fibroin can be obtained from, for example, a cloned gene sequence for the naturally derived fibroin by substituting one or a plurality of hydrophilic amino acid residues (for example, amino acid residues having a negative value of hydropathy index) in REP with hydrophobic amino acid residues (for example, amino acid residues having a positive value of hydropathy index) and/or by inserting one or a plurality of hydrophobic amino acid residues into REP. It is also possible to obtain the fifth modified fibroin by, for example, designing an amino acid sequence corresponding to an amino acid sequence in which one or a plurality of hydrophilic amino acid residues in REP in the amino acid sequence of the naturally derived fibroin are substituted by hydrophobic amino acid residues and/or an amino acid sequence in which one or a plurality of hydrophobic amino acid residues are inserted into REP in the amino acid sequence of the naturally derived fibroin and chemically synthesizing a nucleic acid encoding the designed amino acid sequence. In any case, in addition to the modification corresponding to a substitution of one or a plurality of hydrophilic amino acid residues in REP in the amino acid sequence of the naturally derived fibroin with hydrophobic amino acid residues and/or an insertion of one or a plurality of hydrophobic amino acid residues into REP in the amino acid sequence of the naturally derived fibroin, further amino acid sequence modification may be performed, which corresponds to a substitution, a deletion, an insertion, and/or an addition of one or a plurality of amino acid residues.

The fifth modified fibroin contains a domain sequence represented by Formula 1: [(A)ₙ motif-REP]ₘ, and in a case where a total number of amino acid residues included in regions in which an average value of hydropathy indices of four consecutive amino acid residues is 2.6 or higher in all REP's contained in the domain sequence excluding a sequence from the (A)ₙ motif located at the most C-terminal side to the C-terminus of the domain sequence is denoted by p, and a total number of amino acid residues contained in the domain sequence excluding the sequence from the (A)ₙ motif located at the most C-terminal side to the C-terminus of the domain sequence is denoted by q, the fifth modified fibroin may have an amino acid sequence in which p/q is 6.2% or higher.

As the hydropathy index of an amino acid residue, a known index (Hydropathy index: Kyte J & Doolittle R (1982) “A simple method for displaying the hydropathic character of a protein”, J. Mol. Biol., 157, pp. 105-132) is used. Specifically, a hydropathy index (hereinafter, also referred to as “HI”) of each amino acid is indicated in the following Table 1.

TABLE 1

| Amino acid | HI |
|---|---|
| Isoleucine (Ile) | 4.5 |
| Valine (Val) | 4.2 |
| Leucine (Leu) | 3.8 |
| Phenylalanine (Phe) | 2.8 |
| Cysteine (Cys) | 2.5 |
| Methionine (Met) | 1.9 |
| Alanine (Ala) | 1.8 |
| Glycine (Gly) | −0.4 |
| Threonine (Thr) | −0.7 |
| Serine (Ser) | −0.8 |
| Tryptophan (Trp) | −0.9 |
| Tyrosine (Tyr) | −1.3 |
| Proline (Pro) | −1.6 |
| Histidine (His) | −3.2 |
| Asparagine (Asn) | −3.5 |
| Aspartic acid (Asp) | −3.5 |
| Glutamine (Gln) | −3.5 |
| Glutamic acid (Glu) | −3.5 |
| Lysine (Lys) | −3.9 |
| Arginine (Arg) | −4.5 |

The method for calculating p/q will be described in further detail. In the calculation, a domain sequence represented by Formula 1: [(A)ₙ motif-REP]ₘ is used, excluding a sequence from the (A)ₙ motif located at the most C-terminal side to the C-terminus of the domain sequence (hereinafter, referred to as “sequence A”). First, average values of hydropathy indices of four consecutive amino acid residues in all REP's contained in the sequence A are calculated. The average value of hydropathy indices is calculated by dividing the sum of HI's of all amino acid residues included in the four consecutive amino acid residues by 4 (the number of amino acid residues). The average value of hydropathy indices is calculated for every four consecutive amino acid residues (each amino acid residue is used in the calculation of an average value one to four times). Next, regions in which the average value of hydropathy indices of four consecutive amino acid residues is 2.6 or higher are specified. Even in a case where a certain amino acid residue belongs to a plurality of sets of “four consecutive amino acid residues of which the average value of hydropathy indices is 2.6 or higher”, the amino acid residue is included in the region as one amino acid residue. A total number of amino acid residues included in the regions is p. Furthermore, a total number of amino acid residues contained in the sequence A is q.

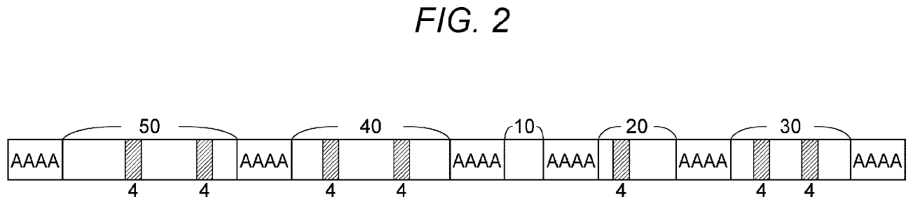

For example, in a case where “four consecutive amino acid residues of which the average value of hydropathy indices is 2.6 or higher” are extracted at 20 locations (without overlaps), 20 sets of four consecutive amino acid residues (without overlaps) are included in the regions in which the average value of hydropathy indices of four consecutive amino acid residues is 2.6 or higher, and p is 20×4=80. Furthermore, in a case where, for example, only one amino acid residue overlaps within two sets of “four consecutive amino acid residues of which the average value of hydropathy indices is 2.6 or higher”, the region in which the average value of hydropathy indices of four consecutive amino acid residues is 2.6 or higher includes seven amino acid residues (p=2×4−1=7. “−1” is a deduction of the overlapping amino acid residue). For example, in a case of the domain sequence shown in FIG. 2, seven sets of “four consecutive amino acid residues of which the average value of hydropathy indices is 2.6 or higher” are present without overlaps, and thus, p is 7×4=28. Furthermore, for example, in the case of the domain sequence shown in FIG. 2, q is 4+50+4+40+4+10+4+20+4+30=170 (the (A)ₙ motif located at the end in the C-terminal side is excluded). Next, p/q (%) can be calculated by dividing p by q. In the case of FIG. 2, 28/170=16.47%.

p/q in the fifth modified fibroin is preferably 6.2% or higher, more preferably 7% or higher, even more preferably 10% or higher, still more preferably 20% or higher, and still even more preferably 30% or higher. The upper limit of p/q is not particularly limited, and may be, for example, 45% or lower.

The fifth modified fibroin can be obtained by, for example, modifying a cloned amino acid sequence of the naturally derived fibroin into an amino acid sequence containing a region in which a hydropathy index is locally high by substituting one or a plurality of hydrophilic amino acid residues (for example, amino acid residues having a negative value of hydropathy index) in REP with hydrophobic amino acid residues (for example, amino acid residues having a positive value of hydropathy index) and/or by inserting one or a plurality of hydrophobic amino acid residues into REP, so that the condition of p/q is satisfied. It is also possible to obtain the fifth modified fibroin by, for example, designing an amino acid sequence satisfying the condition of p/q from the amino acid sequence of the naturally derived fibroin and chemically synthesizing a nucleic acid encoding the designed amino acid sequence. In any case, in addition to the modification corresponding to a substitution of one or a plurality of amino acid residues in REP with amino acid residues having a high hydropathy index and/or an insertion of one or a plurality of amino acid residues having a high hydropathy index into REP, compared to the naturally derived fibroin, further modification may be performed, which corresponds to a substitution, a deletion, an insertion, and/or an addition of one or a plurality of amino acid residues.

The amino acid residue having a high hydropathy index is not particularly limited, and is preferably isoleucine (I), valine (V), leucine (L), phenylalanine (F), cysteine (C), methionine (M), and alanine (A), and more preferably valine (V), leucine (L), and isoleucine (I).

More specific examples of the fifth modified fibroin can include modified fibroin containing (5-i) an amino acid sequence set forth in SEQ ID NO: 19 (Met-PRT720), SEQ ID NO: 20 (Met-PRT665), or SEQ ID NO: 21 (Met-PRT666) or (5-ii) an amino acid sequence having a sequence identity of 90% or higher with the amino acid sequence set forth in SEQ ID NO: 19, SEQ ID NO: 20, or SEQ ID NO: 21.

The modified fibroin of (5-i) will be described. The amino acid sequence set forth in SEQ ID NO: 19 is obtained by inserting amino acid sequences consisting of three amino acid residues (VLI) at two sites for each REP in the amino acid sequence set forth in SEQ ID NO: 7 (Met-PRT410) excluding the terminal domain sequence on the C-terminal side, substituting a part of glutamine (Q) residues with serine (S) residues, and deleting a part of amino acids on the C-terminal side. The amino acid sequence set forth in SEQ ID NO: 20 is obtained by inserting an amino acid sequence consisting of three amino acid residues (VLI) at one site for each REP in the amino acid sequence set forth in SEQ ID NO: 8 (Met-PRT525). The amino acid sequence set forth in SEQ ID NO: 21 is obtained by inserting amino acid sequences consisting of three amino acid residues (VLI) at two sites for each REP in the amino acid sequence set forth in SEQ ID NO: 8.

The modified fibroin of (5-i) may consist of the amino acid sequence set forth in SEQ ID NO: 19, SEQ ID NO: 20, or SEQ ID NO: 21.

The modified fibroin of (5-ii) contains an amino acid sequence having a sequence identity of 90% or higher with the amino acid sequence set forth in SEQ ID NO: 19, SEQ ID NO: 20, or SEQ ID NO: 21. The modified fibroin of (5-ii) is also a protein containing a domain sequence represented by Formula 1: [(A)ₙ motif-REP]ₘ. It is preferable that the sequence identity is 95% or higher.

The modified fibroin of (5-ii) has a sequence identity of 90% or higher with the amino acid sequence set forth in SEQ ID NO: 19, SEQ ID NO: 20, or SEQ ID NO: 21, and in a case where a total number of amino acid residues included in regions in which an average value of hydropathy indices of four consecutive amino acid residues is 2.6 or higher in all REP's contained in the domain sequence excluding a sequence from the (A)ₙ motif located at the most C-terminal side the C-terminus of the domain sequence is denoted by p, and a total number of amino acid residues contained in the domain sequence excluding the sequence from the (A)ₙ motif located at the most C-terminal side to the C-terminus of the domain sequence is denoted by q, p/q is preferably 6.2% or higher.

The fifth modified fibroin may contain a tag sequence at one or both of the N-terminus and the C-terminus thereof.

More specific examples of the modified fibroin containing a tag sequence can include modified fibroin containing (5-iii) an amino acid sequence set forth in SEQ ID NO: 22 (PRT720), SEQ ID NO: 23 (PRT665), or SEQ ID NO: 24 (PRT666) or (5-iv) an amino acid sequence having a sequence identity of 90% or higher with the amino acid sequence set forth in SEQ ID NO: 22, SEQ ID NO: 23, or SEQ ID NO: 24.

The amino acid sequences set forth in SEQ ID NO: 22, SEQ ID NO: 23, and SEQ ID NO: 24 are obtained by adding the amino acid sequence set forth in SEQ ID NO: 11 (which contains a His-tag sequence and a hinge sequence) to the N-termini of the amino acid sequences set forth in SEQ ID NO: 19, SEQ ID NO: 20, and SEQ ID NO: 21, respectively.

The modified fibroin of (5-iii) may consist of the amino acid sequence set forth in SEQ ID NO: 22, SEQ ID NO: 23, or SEQ ID NO: 24.

The modified fibroin of (5-iv) contains an amino acid sequence having a sequence identity of 90% or higher with the amino acid sequence set forth in SEQ ID NO: 22, SEQ ID NO: 23, or SEQ ID NO: 24. The modified fibroin of (5-iv) is also a protein containing a domain sequence represented by Formula 1: [(A)ₙ motif-REP]ₘ. It is preferable that the sequence identity is 95% or higher.

The modified fibroin of (5-iv) has a sequence identity of 90% or higher with the amino acid sequence set forth in SEQ ID NO: 22, SEQ ID NO: 23, or SEQ ID NO: 24, and in a case where a total number of amino acid residues included in regions in which an average value of hydropathy indices of four consecutive amino acid residues is 2.6 or higher in all REP's contained in the domain sequence excluding a sequence from the (A)ₙ motif located at the most C-terminal side to the C-terminus of the domain sequence is denoted by p, and a total number of amino acid residues contained in the domain sequence excluding the sequence from the (A)ₙ motif located at the most C-terminal side to the C-terminus of the domain sequence is denoted by q, p/q is preferably 6.2% or higher.

The fifth modified fibroin may contain a secretory signal for releasing a protein produced in a recombinant protein production system to the outside of a host. A sequence of the secretory signal can be suitably set according to the type of the host.

The sixth modified fibroin has an amino acid sequence in which a content of glutamine residues is reduced, compared to the naturally derived fibroin.

It is preferable that the sixth modified fibroin contains at least one motif selected from a GGX motif and a GPGXX motif in the amino acid sequence of REP.

In a case where the sixth modified fibroin contains the GPGXX motif in REP, a content rate of the GPGXX motifs is generally 1% or higher. The content rate of the GPGXX motif may be 5% or higher and is preferably 10% or higher. The upper limit of the content rate of the GPGXX motifs is not particularly limited, and may be 50% or lower or 30% or lower.

In the present specification, the “content rate of the GPGXX motifs” is a value calculated by the following method.

The content rate of the GPGXX motifs in fibroin containing a domain sequence represented by Formula 1: [(A)ₙ motif-REP]ₘ or Formula 2: [(A)ₙ motif-REP]ₘ−(A)ₙ motif (modified fibroin or naturally derived fibroin) is calculated as s/t, in a case where a number which is three times a total number of the GPGXX motifs (that is, corresponding to the total number of G's and P's in the GPGXX motifs) contained in regions of all REP's contained in the domain sequence excluding a sequence from the (A)ₙ motif located at the most C-terminal side to the C-terminus of the domain sequence is denoted by s, and a total number of amino acid residues in all REP's in the domain sequence excluding the sequence from the (A)ₙ motif located at the most C-terminal side to the C-terminus of the domain sequence and further excluding the (A)ₙ motifs is denoted by t.

In the calculation of the content rate of the GPGXX motifs, the “domain sequence excluding a sequence from the (A)ₙ motif located at the most C-terminal side to the C-terminus of the domain sequence” is used to prevent the calculation result of the GPGXX motif content rate from being affected by a sequence having a low correlation with the sequence characteristic of fibroin that may be contained in the “sequence from the (A)ₙ motif located at the most C-terminal side to the C-terminus of the domain sequence” (a sequence corresponding to REP) in a case where m is small (that is, in a case where the domain sequence is short). When a “GPGXX motif” is located at the C-terminus of REP, even in a case where “XX” is, for example, “AA”, the motif is regarded as a “GPGXX motif”.

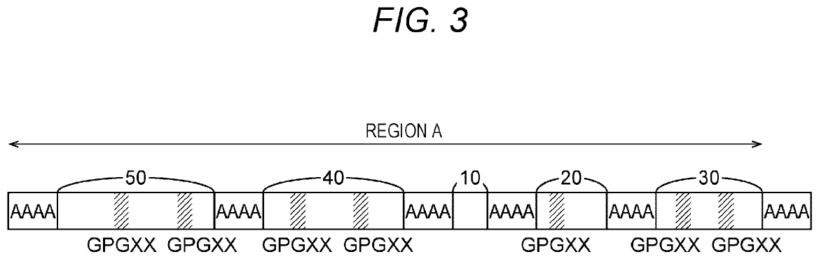

FIG. 3 is a schematic view illustrating a domain sequence of modified fibroin. The method for calculating the content rate of the GPGXX motifs will be specifically described with reference to FIG. 3. First, in the domain sequence of the modified fibroin shown in FIG. 3 (which is the “[(A)ₙ motif-REP]ₘ−(A)ₙ motif” type), all REP's are contained in the “domain sequence excluding the sequence from the (A)ₙ motif located at the most C-terminal side to the C-terminus of the domain sequence” (in FIG. 3, the sequence indicated as a “region A”), and therefore, the number of the GPGXX motifs for calculating s is 7, and s is 7×3=21. Similarly, since all REP's are contained in the “domain sequence excluding the sequence from the (A)ₙ motif located at the most C-terminal side to the C-terminus of the domain sequence” (in FIG. 3, the sequence indicated as the “region A”), the total number t of the amino acid residues in all REP's further excluding the (A)ₙ motifs from the sequence is 50+40+10+20+30=150. Next, s/t (%) can be calculated by dividing s by t, and in the case of the modified fibroin of FIG. 3, s/t is 21/150=14.0%.

The content rate of glutamine residues in the sixth modified fibroin is preferably 9% or lower, more preferably 7% or lower, even more preferably 4% or lower, and particularly preferably 0%.

In the present specification, the “content rate of glutamine residues” is a value calculated by the following method.

The content rate of glutamine residues in fibroin containing a domain sequence represented by Formula 1: [(A)ₙ motif-REP]ₘ or Formula 2: [(A)ₙ motif-REP]ₘ−(A)ₙ motif (modified fibroin or naturally derived fibroin) is calculated as u/t, in a case where a total number of glutamine residues contained in regions of all REP's contained in the domain sequence excluding a sequence from the (A)ₙ motif located at the most C-terminal side to the C-terminus of the domain sequence (a sequence corresponding to the “region A” in FIG. 3) is denoted by u, and a total number of amino acid residues in all REP's in the domain sequence excluding the sequence from the (A)ₙ motif located at the most C-terminal side to the C-terminus of the domain sequence and further excluding the (A)ₙ motifs is denoted by t. In the calculation of the content rate of glutamine residues, the “domain sequence excluding a sequence from the (A)ₙ motif located at the most C-terminal side to the C-terminus of the domain sequence” is used for the same reason described above.

The domain sequence of the sixth modified fibroin may have an amino acid sequence corresponding to an amino acid sequence in which one or a plurality of glutamine residues in REP are deleted or substituted by other amino acid residues, compared to the naturally derived fibroin.

“Other amino acid residues” may be any amino acid residues other than glutamine residues, and an amino acid residue having a higher hydropathy index than the glutamine residue is preferred. The hydropathy indices of amino acid residues are as indicated in Table 1.

As indicated in Table 1, examples of the amino acid residue having a higher hydropathy index than the glutamine residue can include an amino acid residue selected from isoleucine (I), valine (V), leucine (L), phenylalanine (F), cysteine (C), methionine (M) alanine (A), glycine (G), threonine (T), serine (S), tryptophan (W), tyrosine (Y), proline (P), and histidine (H). Among these, an amino acid residue selected from isoleucine (I), valine (V), leucine (L), phenylalanine (F), cysteine (C), methionine (M), and alanine (A) is more preferred, and an amino acid residue selected from isoleucine (I), valine (V), leucine (L), and phenylalanine (F) is even more preferred.

In the sixth modified fibroin, hydrophobicity of REP is preferably −0.8 or higher, more preferably −0.7 or higher, even more preferably 0 or higher, still more preferably 0.3 or higher, and particularly preferably 0.4 or higher. The upper limit of the hydrophobicity of REP is not particularly limited, and may be 1.0 or lower or 0.7 or lower.

In the present specification, the “hydrophobicity of REP” is a value calculated by the following method. The hydrophobicity of REP in fibroin containing a domain sequence represented by Formula 1: [(A)ₙ motif-REP]ₘ or Formula 2: [(A)ₙ motif-REP]ₘ−(A)ₙ motif (modified fibroin or naturally derived fibroin) is calculated as v/t, in a case where a sum of hydropathy indices of all amino acid residues in regions of all REP's contained in the domain sequence excluding a sequence from the (A)ₙ motif located at the most C-terminal side to the C-terminus of the domain sequence (a sequence corresponding to the “region A” in FIG. 3) is denoted by v, and a total number of amino acid residues in all REP's in the domain sequence excluding the sequence from the (A)ₙ motif located at the most C-terminal side to the C-terminus of the domain sequence and further excluding the (A)ₙ motifs is denoted by t. In the calculation of the hydrophobicity of REP, the “domain sequence excluding a sequence from the (A)ₙ motif located at the most C-terminal side to the C-terminus of the domain sequence” is used for the same reason described above.

In addition to the modification corresponding to a deletion of one or a plurality of glutamine residues in REP and/or a substitution of one or a plurality of glutamine residues in REP with other amino acid residues, compared to the naturally derived fibroin, further amino acid sequence modification may be performed on the domain sequence of the sixth modified fibroin, which corresponds to a substitution, a deletion, an insertion, and/or an addition of one or a plurality of amino acid residues.

The sixth modified fibroin can be obtained from, for example, a cloned gene sequence for the naturally derived fibroin by deleting one or a plurality of glutamine residues in REP and/or substituting one or a plurality of glutamine residues in REP with other amino acid residues. It is also possible to obtain the sixth modified fibroin by, for example, designing an amino acid sequence corresponding to an amino acid sequence in which one or a plurality of glutamine residues in REP in the amino acid sequence of the naturally derived fibroin are deleted and/or one or a plurality of glutamine residues in REP in the amino acid sequence of the naturally derived fibroin are substituted by other amino acid residues and chemically synthesizing a nucleic acid encoding the designed amino acid sequence.

More specific examples of the sixth modified fibroin can include modified fibroin containing (6-i) an amino acid sequence set forth in SEQ ID NO: 25 (Met-PRT888), SEQ ID NO: 26 (Met-PRT965), SEQ ID NO: 27 (Met-PRT889), SEQ ID NO: 28 (Met-PRT916), SEQ ID NO: 29 (Met-PRT918), SEQ ID NO: 30 (Met-PRT699), SEQ ID NO: 31 (Met-PRT698), SEQ ID NO: 32 (Met-PRT966), SEQ ID NO: 41 (Met-PRT917), or SEQ ID NO: 42 (Met-PRT1028) or modified fibroin containing (6-ii) an amino acid sequence having a sequence identity of 90% or higher with the amino acid sequence set forth in SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 41, or SEQ ID NO: 42.

The modified fibroin of (6-i) will be described. The amino acid sequence set forth in SEQ ID NO: 25 is obtained by substituting all QQ's in the amino acid sequence set forth in SEQ ID NO: 7 (Met-PRT410) with VL's. The amino acid sequence set forth in SEQ ID NO: 26 is obtained by substituting all QQ's in the amino acid sequence set forth in SEQ ID NO: 7 with TS's and substituting the remaining Q's with A's. The amino acid sequence set forth in SEQ ID NO: 27 is obtained by substituting all QQ's in the amino acid sequence set forth in SEQ ID NO: 7 with VL's and substituting the remaining Q's with I's. The amino acid sequence set forth in SEQ ID NO: 28 is obtained by substituting all QQ's in the amino acid sequence set forth in SEQ ID NO: 7 with VI's and substituting the remaining Q's with L's. The amino acid sequence set forth in SEQ ID NO: 29 is obtained by substituting all QQ's in the amino acid sequence set forth in SEQ ID NO: 7 with VF's and substituting the remaining Q's with I's.

The amino acid sequence set forth in SEQ ID NO: 30 is obtained by substituting all QQ's in the amino acid sequence set forth in SEQ ID NO: 8 (Met-PRT525) with VL's. The amino acid sequence set forth in SEQ ID NO: 31 is obtained by substituting all QQ's in the amino acid sequence set forth in SEQ ID NO: 8 with VL's and substituting the remaining Q's with I's.

The amino acid sequence set forth in SEQ ID NO: 32 is obtained by substituting all QQ's with VF's in a sequence in which a region of 20 domain sequences existing in the amino acid sequence set forth in SEQ ID NO: 7 (Met-PRT410) is repeated twice and substituting the remaining Q's with I's.

The amino acid sequence set forth in SEQ ID NO: 41 (Met-PRT917) is obtained by substituting all QQ's in the amino acid sequence set forth in SEQ ID NO: 7 with LI's and substituting the remaining Q's with V's. The amino acid sequence set forth in SEQ ID NO: 42 (Met-PRT1028) is obtained by substituting all QQ's in the amino acid sequence set forth in SEQ ID NO: 7 with IF's and substituting the remaining Q's with T's.

The content rate of glutamine residues in each of the amino acid sequences set forth in SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 41, and SEQ ID NO: 42 is 9% or lower (Table 2).

TABLE 2

| | Content | Content | |
|---|---|---|---|
| | rate of | rate of | Hydro- |
| | glutamine | GPGXX | phobicity |
| Modified fibroin | residues | motifs | of REP |
| Met-PRT410 (SEQ ID NO: 7) | 17.7% | 27.9% | −1.52 |
| Met-PRT888 (SEQ ID NO: 25) | 6.3% | 27.9% | −0.07 |
| Met-PRT965 (SEQ ID NO: 26) | 0.0% | 27.9% | −0.65 |
| Met-PRT889 (SEQ ID NO: 27) | 0.0% | 27.9% | 0.35 |
| Met-PRT916 (SEQ ID NO: 28) | 0.0% | 27.9% | 0.47 |
| Met-PRT918 (SEQ ID NO: 29) | 0.0% | 27.9% | 0.45 |
| Met-PRT699 (SEQ ID NO: 30) | 3.6% | 26.4% | −0.78 |
| Met-PRT698 (SEQ ID NO: 31) | 0.0% | 26.4% | −0.03 |
| Met-PRT966 (SEQ ID NO: 32) | 0.0% | 28.0% | 0.35 |
| Met-PRT917 (SEQ ID NO: 41) | 0.0% | 27.9% | 0.46 |
| Met-PRT1028 (SEQ ID NO: 42) | 0.0% | 28.1% | 0.05 |

The modified fibroin of (6-i) may consist of the amino acid sequence set forth in SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 41, or SEQ ID NO: 42.

The modified fibroin of (6-ii) contains an amino acid sequence having a sequence identity of 90% or higher with the amino acid sequence set forth in SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 41, or SEQ ID NO: 42. The modified fibroin of (6-ii) is also a protein containing a domain sequence represented by Formula 1: [(A)ₙ motif-REP]ₘ or Formula 2: [(A)ₙ motif-REP]ₘ−(A)ₙ motif. It is preferable that the sequence identity is 95% or higher.

The content rate of glutamine residues in the modified fibroin of (6-ii) is preferably 9% or lower. Furthermore, the content rate of the GPGXX motifs in the modified fibroin of (6-ii) is preferably 10% or higher.

The sixth modified fibroin may contain a tag sequence at one or both of the N-terminus and the C-terminus thereof. By containing a tag sequence, isolation, immobilization, detection, visualization, and the like of the modified fibroin become possible.

More specific examples of the modified fibroin containing a tag sequence can include modified fibroin containing (6-iii) an amino acid sequence set forth in SEQ ID NO: 33 (PRT888), SEQ ID NO: 34 (PRT965), SEQ ID NO: 35 (PRT889), SEQ ID NO: 36 (PRT916), SEQ ID NO: 37 (PRT918), SEQ ID NO: 38 (PRT699), SEQ ID NO: 39 (PRT698), SEQ ID NO: 40 (PRT966), SEQ ID NO: 43 (PRT917), or SEQ ID NO: 44 (PRT1028) or modified fibroin containing (6-iv) an amino acid sequence having a sequence identity of 90% or higher with the amino acid sequence set forth in SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 43, or SEQ ID NO: 44.

The amino acid sequences set forth in SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 43, and SEQ ID NO: 44 are obtained by adding the amino acid sequence set forth in SEQ ID NO: 11 (which contains a His-tag sequence and a hinge sequence) to the N-termini of the amino acid sequences set forth in SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 41, and SEQ ID NO: 42, respectively. Since only the tag sequence is added to the N-termini, the content rates of glutamine residues do not change, and the content rate of glutamine residues in each of the amino acid sequences set forth in SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 43, and SEQ ID NO: 44 is 9% or lower (Table 3).

TABLE 3

| | Content | Content | |
|---|---|---|---|
| | rate of | rate of | Hydro- |
| | glutamine | GPGXX | phobicity |
| Modified fibroin | residues | motifs | of REP |
| PRT888 (SEQ ID NO: 33) | 6.3% | 27.9% | −0.07 |
| PRT965 (SEQ ID NO: 34) | 0.0% | 27.9% | −0.65 |
| PRT889 (SEQ ID NO: 35) | 0.0% | 27.9% | 0.35 |
| PRT916 (SEQ ID NO: 36) | 0.0% | 27.9% | 0.47 |
| PRT918 (SEQ ID NO: 37) | 0.0% | 27.9% | 0.45 |
| PRT699 (SEQ ID NO: 38) | 3.6% | 26.4% | −0.78 |
| PRT698 (SEQ ID NO: 39) | 0.0% | 26.4% | −0.03 |
| PRT966 (SEQ ID NO: 40) | 0.0% | 28.0% | 0.35 |
| PRT917 (SEQ ID NO: 43) | 0.0% | 27.9% | 0.46 |
| PRT1028 (SEQ ID NO: 44) | 0.0% | 28.1% | 0.05 |

The modified fibroin of (6-iii) may consist of the amino acid sequence set forth in SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 43, or SEQ ID NO: 44.

The modified fibroin of (6-iv) contains an amino acid sequence having a sequence identity of 90% or higher with the amino acid sequence set forth in SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 43, or SEQ ID NO: 44. The modified fibroin of (6-iv) is also a protein containing a domain sequence represented by Formula 1: [(A)ₙ motif-REP]ₘ or Formula 2: [(A)/ₙ motif-REP]ₘ−(A)ₙ motif. It is preferable that the sequence identity is 95% or higher.

The content rate of glutamine residues in the modified fibroin of (6-iv) is preferably 9% or lower. Furthermore, the content rate of the GPGXX motifs in the modified fibroin of (6-iv) is preferably 10% or higher.

The sixth modified fibroin may contain a secretory signal for releasing a protein produced in a recombinant protein production system to the outside of a host. A sequence of the secretory signal can be suitably set according to the type of the host.

The modified fibroin may have at least two or more of the characteristics among the characteristics of the first modified fibroin, the second modified fibroin, the third modified fibroin, the fourth modified fibroin, the fifth modified fibroin, and the sixth modified fibroin.

The modified fibroin may be hydrophilic modified fibroin or hydrophobic modified fibroin. The hydrophobic modified fibroin is modified fibroin of which a value calculated by obtaining a sum of hydropathy indices (HI's) of all amino acid residues constituting the modified fibroin and then dividing the sum by a total number of amino acid residues (average HI) is 0 or larger. The hydropathy indices are as indicated in Table 1. In addition, the hydrophilic modified fibroin is modified fibroin of which the average HI is lower than 0.

Examples of the hydrophobic modified fibroin can include the sixth modified fibroin described above. More specific examples of the hydrophobic modified fibroin include modified fibroin containing an amino acid sequence set forth in SEQ ID NO: 27, SEQ ID NO: 28, SEQ ID NO: 29, SEQ ID NO: 30, SEQ ID NO: 31, SEQ ID NO: 32, SEQ ID NO: 33, or SEQ ID NO: 43, or an amino acid sequence set forth in SEQ ID NO: 35, SEQ ID NO: 37, SEQ ID NO: 38, SEQ ID NO: 39, SEQ ID NO: 40, SEQ ID NO: 41, or SEQ ID NO: 44.

Examples of the hydrophilic modified fibroin can include the first modified fibroin, the second modified fibroin, the third modified fibroin, the fourth modified fibroin, and the fifth modified fibroin described above. More specific examples of the hydrophilic modified fibroin can include modified fibroin containing an amino acid sequence set forth in SEQ ID NO: 4, an amino acid sequence set forth in SEQ ID NO: 6, SEQ ID NO: 7, SEQ ID NO: 8, or SEQ ID NO: 9, an amino acid sequence set forth in SEQ ID NO: 13, SEQ ID NO: 11, SEQ ID NO: 14, or SEQ ID NO: 15, an amino acid sequence set forth in SEQ ID NO: 18, SEQ ID NO: 7, SEQ ID NO: 8, or SEQ ID NO: 9, an amino acid sequence set forth in SEQ ID NO: 17, SEQ ID NO: 11, SEQ ID NO: 14, or SEQ ID NO: 15, or an amino acid sequence set forth in SEQ ID NO: 19, SEQ ID NO: 20, or SEQ ID NO: 21.

(Method for Producing Protein)

A protein can be produced by, for example, expressing a nucleic acid encoding the protein using a host transformed with an expression vector having the nucleic acid sequence and one or a plurality of regulatory sequences operatively linked to the nucleic acid sequence.

A method for producing a gene encoding the protein is not particularly limited. For example, the gene can be produced using a gene encoding a natural structural protein by a method of performing amplification by polymerase chain reaction (PCR) to clone the gene or by chemical synthesis. The method for chemically synthesizing a gene is not particularly limited, and for example, a gene can be chemically synthesized by a method of linking, by PCR or the like, oligonucleotides automatically synthesized with AKTA oligopilot plus 10/100 (GE Healthcare Japan Corporation) or the like based on amino acid sequence information of the structural protein obtained from the NCBI web database or the like. In this case, in order to allow easy purification or confirmation of the protein, a gene may be synthesized which encodes a protein consisting of an amino acid sequence which includes the above amino acid sequence and an amino acid sequence consisting of a start codon and a His10-tag added to the N-terminus thereof.

The regulatory sequence is a sequence that controls the expression of a protein in a host (for example, a promoter, an enhancer, a ribosome binding sequence, a transcription termination sequence, and the like), and can be appropriately selected according to the type of the host. As a promoter, an inducible promoter that can function in a host cell and induce the expression of the protein may be used. The inducible promoter is a promoter that can control transcription by the presence of an inducer (expression inducing agent), absence of a repressor molecule, or a physical factor such as an increase or decrease in a temperature, osmotic pressure, or a pH value.

The type of the expression vector can be appropriately selected according to the type of the host, and examples thereof include a plasmid vector, a virus vector, a cosmid vector, a fosmid vector, an artificial chromosome vector, and the like. An expression vector which is capable of autonomously replicating in the host cell or integrating into the host chromosome and has a promoter at a site where the nucleic acid encoding the protein can be transcribed is suitably used.

As the host, both a prokaryote and a eukaryote such as yeast, filamentous fungi, insect cells, animal cells, and plant cells can be suitably used.

Preferable examples of the prokaryote can include bacteria belonging to the genera Escherichia, Brevibacillus, Serratia, Bacillus, Microbacterium, Brevibacterium, Corynebacterium, and Pseudomonas. Examples of the microorganism belonging to the genus Escherichia can include Escherichia coli. Examples of the microorganism belonging to the genus Brevibacillus can include Brevibacillus agri. Examples of the microorganism belonging to the genus Serratia can include Serratia liquefaciens. Examples of the microorganism belonging to the genus Bacillus can include Bacillus subtilis. Examples of the microorganism belonging to the genus Microbacterium can include microbacterium ammoniaphilum. Examples of the microorganism belonging to the genus Brevibacterium can include Brevibacterium divaricatum. Examples of the microorganism belonging to the genus Corynebacterium can include Corynebacterium ammoniagenes. Examples of the microorganism belonging to the genus Pseudomonas can include Pseudomonas putida.

In a case where a prokaryote is used as the host, examples of the vector for introducing the nucleic acid encoding the recombinant protein can include pBTrp2 (manufactured by Boehringer Mannheim GmbH), pGEX (manufactured by Pharmacia), pUC18, pBluescriptII, pSupex, pET22b, pCold, pUB110, and pNCO2 (JP 2002-238569 A).

Examples of the eukaryotic host can include yeast and filamentous fungi (mold or the like). Examples of the yeast can include yeasts belonging to the genera Saccharomyces, Pichia, and Schizosaccharomyces. Examples of the filamentous fungi can include filamentous fungi belonging to the genera Aspergillus, Penicillium, and Trichoderma.

In a case where a eukaryote is used as the host, examples of the vector for introducing the nucleic acid encoding the protein can include YEP13 (ATCC37115) and YEp24 (ATCC37051). Any method can be used as a method for introducing the expression vector into the host cell, as long as it is a method for introducing DNA into the host cell. For example, a method using calcium ions [Proc. Natl. Acad. Sci. USA, 69, 2110 (1972)], an electroporation method, a spheroplast method, a protoplast method, a lithium acetate method, a competent method, or the like can be used.

As a method for expressing the nucleic acid by the host transformed with the expression vector, secretory production, fusion protein expression, or the like can be performed based on the method described in Molecular Cloning, 2^{nd} edition, in addition to direct expression.

The protein can be produced by, for example, culturing the host transformed with the expression vector in a culture medium, producing and accumulating the protein in the culture medium, and collecting the protein from the culture medium. A method for culturing the host in the culture medium can be performed according to a method generally used for culturing a host.

In a case where the host is a prokaryote such as Escherichia coli or a eukaryote such as yeast, any one of a natural medium and a synthetic medium may be used as the culture medium, as long as it is a medium containing a carbon source, a nitrogen source, inorganic salts, and the like that can be assimilated by the host and capable of efficiently culturing the host.

Any carbon source that can be assimilated by the transformed microorganism may be used, and for example, carbohydrates such as glucose, fructose, sucrose, molasses containing glucose, fructose, and sucrose, starch, and a starch hydrolyzate, organic acids such as acetic acid and propionic acid, and alcohols such as ethanol and propanol can be used. As the nitrogen source, for example, ammonia, ammonium salts of an inorganic acid or organic acid, such as ammonium chloride, ammonium sulfate, ammonium acetate, and ammonium phosphate, other nitrogen-containing compounds, peptone, a meat extract, a yeast extract, corn steep liquor, a casein hydrolyzate, soybean meal and a soybean meal hydrolyzate, and various fermentative bacteria cells and digests thereof can be used. As the inorganic salt, for example, monopotassium phosphate, dipotassium phosphate, magnesium phosphate, magnesium sulfate, sodium chloride, iron(II) sulfate, manganese sulfate, copper sulfate, and calcium carbonate can be used.

A prokaryote such as Escherichia coli or a eukaryote such as yeast can be cultured under, for example, an aerobic condition such as shaking culture or deep aeration stirring culture. A culture temperature is, for example, 15° C. to 40° C. Culture time is generally 16 hours to 7 days. It is preferable that a pH of the culture medium is maintained at 3.0 to 9.0 during the culture. The pH of the culture medium can be adjusted using an inorganic acid, an organic acid, an alkaline solution, urea, calcium carbonate, ammonia, or the like.

In addition, an antibiotic such as ampicillin and tetracycline may be added to the culture medium during the culture as necessary. When culturing a microorganism transformed with an expression vector using an inducible promoter as the promoter, an inducer may be added to the medium as necessary. For example, when culturing a microorganism transformed with an expression vector using a lac promoter, isopropyl-β-D-thiogalactopyranoside may be added to the medium, and when culturing a microorganism transformed with an expression vector using a trp promoter, indoleacrylic acid may be added to the medium.

Isolation and purification of the expressed protein can be performed by a method that is generally used. For example, in a case where the protein is expressed in a state of being dissolved in the cells, the host cells are collected by centrifugation after the termination of the culture and suspended in an aqueous buffer. Then, the host cells are disrupted by an ultrasonic disintegrator, a French press, a Manton-Gaulin homogenizer, a Dyno-mill, or the like, and a cell-free extract is obtained. A method that is generally used in isolation and purification of proteins from a supernatant obtained by centrifugation of the cell-free extract, that is, a method such as a solvent extraction method, a salting-out method using ammonium sulfate, a desalination method, a precipitation method using an organic solvent, an anion exchange chromatography method using a resin such as diethylaminoethyl (DEAE)-Sepharose and DIAION HPA-75 (manufactured by Mitsubishi Kasei Corporation), a cation exchange chromatography method using a resin such as S-Sepharose FF (manufactured by Pharmacia), a hydrophobic chromatography method using a resin such as butyl-Sepharose and phenyl-Sepharose, a gel filtration method using a molecular sieve, an affinity chromatography method, a chromatofocusing method, and an electrophoresis method such as isoelectric focusing can be used alone or in combination to obtain a purified preparation.

Furthermore, in a case where the protein is expressed by forming an insoluble matter in the cells, the host cells are collected in the same manner, and then disrupted and subjected to centrifugation, thereby collecting the insoluble matter of the protein as a precipitated fraction. The insoluble matter of the protein thus collected can be solubilized by a protein denaturant. After the operation, a purified preparation of the protein can be obtained by the same isolation and purification methods as those described above. In a case where the protein is secreted outside the cells, the protein can be collected from a culture supernatant. That is, a culture supernatant is acquired by treating the culture by a method such as centrifugation, and a purified preparation can be obtained from the culture supernatant using the same isolation and purification methods as those described above.

[Hydrolysis Step]

The production method according to the present embodiment includes a step of bringing a raw material molded article containing a protein in which a hydroxyl group is esterified into contact with an acidic or basic medium in a state of applying a tensile force, thus hydrolyzing an ester group (hereinafter, referred to as a “hydrolysis step”). It is considered that, since penetration of the medium into the raw material molded article is suppressed by performing the hydrolysis in the state of applying a tensile force, ester groups inside the raw material molded article are difficult to be removed or reduced, and only ester groups on a surface of the raw material molded article are easily removed or reduced. It is possible to fully expect that foul odor generation or the like caused by the hydrolysis of ester groups in a molded article is suppressed by simply removing or reducing only the ester groups on the surface of the raw material molded article. In addition, it is speculated that a cause of strength reduction in the protein molded article is hydrolysis of a protein backbone inside the raw material molded article. It is thus considered that the problem caused by the esterification of the hydroxyl groups contained in the protein can be solved while maintaining a sufficient strength of the protein molded article by performing the hydrolysis in the state of applying a tensile force. Furthermore, toughness of the protein molded article (an area of a region surrounded by a stress (strength) and a degree of elongation in a stress (strength)-degree of elongation curve) is improved by performing the hydrolysis in the state of applying a tensile force.

In the present embodiment, the amount of the tensile force is preferably an amount in which the raw material molded article does not shrink by the contact with the medium (for example, an aqueous solution). The tensile force can be applied by, for example, a filament winding (FW) method, and can be adjusted by an applied load.

The tensile force at which the raw material molded article does not shrink by the contact with the medium can be determined by, for example, bringing the raw material molded article into contact with the medium in a state of applying various tensile forces and obtaining a tensile force at which a rate of change in the size of the raw material molded article (for example, a fiber length in a case of a fiber, a volume in a case of a heat compression molded article, a porous body, or a gel, and an area in a case of a film) immediately after the contact with the medium is 87 to 113%, preferably 90 to 110%, more preferably 93 to 107%, and particularly preferably 95 to 105%.

For example, in a case where the raw material molded article is a fiber (raw material fiber), the tensile force during the treatment is preferably 150 MPa or less, more preferably 125 MPa or less, even more preferably 100 MPa or less, and most preferably 75 MPa or less.

In the present embodiment, the medium is an acidic or basic medium. The medium may be an alcohol-based solvent or an amine-based organic solvent in which ester can be solvolyzed. However, from the viewpoint that a hydrolysis treatment which is a type of the solvolysis can be easily performed, the medium desirably contains water, and an aqueous solution and an aqueous vapor are more desirable as the medium. An alkaline aqueous solution (medium) may be any alkaline aqueous solution as long as it exhibits alkaline properties. For example, an alkaline aqueous solution with a pH higher than 7 may be used, and an alkaline aqueous solution with a pH lower than 12 is preferred from the viewpoint of suppressing hydrolysis of a molecular chain and a side reaction. An acidic aqueous solution (medium) may be any acidic aqueous solution as long as it exhibits acidic properties. For example, an acidic aqueous solution with a pH lower than 7 may be used, and an acidic aqueous solution with a pH of 1 or higher is preferred from the viewpoint of suppressing hydrolysis of a molecular chain and a side reaction.

In the present embodiment, examples of a method for performing the hydrolysis can include a method of bringing the raw material molded article into contact with the acidic or alkaline aqueous solution. In this case, the amount of an acidic substance or an alkaline substance is preferably 0.1% by mass or more and more preferably 1.0% by mass or more, with respect to the total amount of a protein solution.

The acidic substance that can be used in the hydrolysis may be, but not particularly limited to, either an inorganic acid or an organic acid. Examples of the inorganic acid include hydrochloric acid, sulfuric acid, nitric acid, phosphoric acid, and boric acid. Examples of the organic acid include carboxylic acid and sulfonic acid. Examples of the carboxylic acid include a monocarboxylic acid such as formic acid, acetic acid, dichloroacetic acid, trifluoroacetic acid, propionic acid, butanoic acid, isobutyric acid, pentanoic acid, caproic acid, caprylic acid, capric acid, and benzoic acid, a saturated aliphatic carboxylic acid such as capric acid, lauric acid, myristic acid, palmitic acid, stearic acid, arachidic acid, behenic acid, lignoceric acid, cerotic acid, montanic acid, melissic acid, and ceroplastic acid, an unsaturated aliphatic carboxylic acid such as undecylenic acid, oleic acid, elaidic acid, cetoleic acid, erucic acid, brassidic acid, sorbic acid, linoleic acid, linolenic acid, arachidonic acid, propiolic acid, and stearolic acid, and a dicarboxylic acid such as oxalic acid, malonic acid, succinic acid, glutaric acid, adipic acid, pimelic acid, suberic acid, azelaic acid, sebacic acid, undecanoic acid, brassylic acid, maleic acid, fumaric acid, and glutaconic acid. The carboxylic acid may be in a form of an acid anhydride or an acid chloride. Examples of the sulfonic acid include methanesulfonic acid, trifluoromethanesulfonic acid, benzenesulfonic acid, and p-toluenesulfonic acid.

The alkaline substance that can be used in the hydrolysis is not particularly limited as long as it is soluble in water, and may be either an inorganic base or an organic base. The inorganic base is not particularly limited as long as it is soluble in water. Examples of the inorganic base include potassium hydroxide, sodium hydroxide, sodium bicarbonate, and sodium carbonate. Examples of the organic base include ammonia, alkylamine such as methylamine, dimethylamine, trimethylamine, ethylamine, diethylamine, and triethylamine, and primary to tertiary amines such as aminoethanol, methylaminoethanol, dimethylaminoethanol, ethylaminoethanol, diethylaminoethanol, and diethanolamine.

The hydrolysis of the ester group is an equilibrium reaction. Although an acid that is the same as the acid produced when the ester group is dissociated can be used as the acid needed for the hydrolysis, it is preferable not to use such an acid.

In the present embodiment, the hydrolysis proceeds under an acidic condition or an alkaline condition. An acidic condition in which a pH is 1 to 6 or an alkaline condition in which a pH is 8 to 14 is preferred, and the pH can be adjusted by the acidic substance or alkaline substance that is used.

In the present embodiment, in a case where an alkaline aqueous solution is used, the pH is preferably higher than 8 and preferably lower than 12, due to the reasons described below.

In a case where the alkaline aqueous solution is used, after the carboxylic acid is dissociated by the hydrolysis, a carboxylic acid anion is formed. In this case, the electrophilicity is lost, and thus, a reverse reaction is less likely to occur. From this point, the aqueous solution (medium) is preferably an alkaline aqueous solution, rather than an acidic aqueous solution. The pH of the alkaline aqueous solution is preferably 8 to 14, from the viewpoint of implementing high reactivity without heating, and the pH of the alkaline aqueous solution is more preferably higher than 8, from the viewpoint of the reaction rate of the hydrolysis. In addition, in a case where a raw material composition is a molded article, the pH is preferably lower than 12 from the viewpoint of minimizing the breakage of a molecular chain such as dispersion destruction and hydrolysis of an amide in a strongly alkaline environment.

Since the hydrolysis an ester group rapidly proceeds in the acidic or alkaline aqueous solution, reaction time is preferably longer than 1 minute, from the viewpoint of sufficiently removing the ester group, however, the reaction time is not limited thereto.

In the present embodiment, a temperature at which the hydrolysis is performed is not particularly limited, and may be, for example, 5° C. or higher, 10° C. or higher, 20° C. or higher, 30° C. or higher, or 80° C. or higher. Furthermore, the temperature at which the hydrolysis is performed may be, for example, 90° C. or lower, 80° C. or lower, 50° C. or lower, 40° C. or lower, or 35° C. or lower.

In the present embodiment, the acidic substance or the alkaline substance may remain on the molded article that has been taken out of the aqueous solution used in the hydrolysis, and the acidic substance or the alkaline substance may cause the breakage of a molecular chain. For the purpose of preventing such breakage of a molecular chain, the method for producing a protein molded article may further include a step of removing the acidic substance or the alkaline substance remaining on the molded article. Examples of the step of removing the acidic substance or the alkaline substance remaining on the molded article include a step of washing the molded article with water.

(Method for Producing Raw Material Molded Article)

In the present embodiment, a method for producing a raw material molded article is not particularly limited, and may be, for example, a method including each of the following steps.

[Dissolution Step]

A dissolution step is a step of dissolving a protein in a solvent (for example, a carboxylic acid such as formic acid) to obtain a protein solution.

In the dissolution step, as the protein to be dissolved (hereinafter, referred to as “hereinafter, a target protein”), a purified protein may be used, or a protein in host cells in which the protein is expressed (recombinant protein) may be used. The purified protein may be a protein purified from host cells in which the protein is expressed. In a case where the protein in the host cells is dissolved as the target protein, the host cells are brought into contact with a solvent to dissolve the protein in the host cells in the solvent. Any host cells may be used as long as they are cells in which the target protein is expressed, and may be, for example, intact cells or cells subjected to a treatment such as a disruption treatment. Alternatively, the cells may be cells subjected to a simple purification treatment in advance.

A method for purifying a protein from host cells in which the protein is expressed is not particularly limited, and, for example, the methods disclosed in JP 6077570 B2 and JP 6077569 B2 can be used.

In the dissolution step, in a case where a carboxylic acid such as formic acid is used as the solvent, an esterified protein is produced by a dehydration condensation reaction between a hydroxyl group in the protein and the carboxylic acid. As the carboxylic acid, it is possible to use, for example, a monocarboxylic acid such as formic acid, acetic acid, dichloroacetic acid, trifluoroacetic acid, propionic acid, butanoic acid, isobutyric acid, pentanoic acid, caproic acid, caprylic acid, capric acid, and benzoic acid, a saturated aliphatic carboxylic acid such as capric acid, lauric acid, myristic acid, palmitic acid, stearic acid, arachidic acid, behenic acid, lignoceric acid, cerotic acid, montanic acid, melissic acid, and ceroplastic acid, an unsaturated aliphatic carboxylic acid such as undecylenic acid, oleic acid, elaidic acid, cetoleic acid, erucic acid, brassidic acid, sorbic acid, linoleic acid, linolenic acid, arachidonic acid, propiolic acid, and stearolic acid, and a dicarboxylic acid such as oxalic acid, malonic acid, succinic acid, glutaric acid, adipic acid, pimelic acid, suberic acid, azelaic acid, sebacic acid, undecanoic acid, brassylic acid, maleic acid, fumaric acid, and glutaconic acid. The carboxylic acid may be in a form of an acid anhydride or an acid chloride.

The dissolution step may be performed at room temperature, or may be performed by dissolving the protein in the solvent while holding the temperature at various heating temperatures. Time for holding the heating temperature is not particularly limited, and may be 10 minutes or longer, and in consideration of industrial production, 10 to 120 minutes is preferred, 10 to 60 minutes is more preferred, and 10 to 30 minutes is even more preferred. The time for holding the heating temperature may be appropriately set under a condition in which the protein is sufficiently dissolved and impurities (other than the target protein) are less dissolved.

An addition amount of the solvent added to dissolve the protein is not particularly limited as long as it is the amount in which the protein can be dissolved.

In a case where a purified protein is dissolved, the addition amount of the solvent may be 1 to 100 times, 1 to 50 times, 1 to 25 times, 1 to 10 times, or 1 to 5 times, in terms of a ratio (volume (mL)/weight (g)) of a volume (mL) of the solvent to a weight (g) of the protein (dry powder containing the protein).

In a case where a protein in host cells in which the protein is expressed is dissolved, the addition amount of the solvent may be 1 to 100 times, 1 to 50 times, 1 to 25 times, 1 to 10 times, or 1 to 5 times, in terms of a ratio (volume (mL)/weight (g)) of the solvent (mL) to a weight (g) of the host cells.

The solvent may contain an inorganic salt. Solubility of the protein can be increased by adding the inorganic salt to the solvent.

Examples of the inorganic salt that can be added to the solvent can include an alkali metal halide, an alkaline earth metal halide, an alkaline earth metal nitrate, a thiocyanate, and a perchlorate.

Examples of the alkali metal halide can include potassium bromide, sodium bromide, lithium bromide, potassium chloride, sodium chloride, lithium chloride, sodium fluoride, potassium fluoride, cesium fluoride, potassium iodide, sodium iodide, and lithium iodide.

Examples of the alkaline earth metal halide can include calcium chloride, magnesium chloride, magnesium bromide, calcium bromide, magnesium iodide, and calcium iodide.

Examples of the alkaline earth metal nitrate can include calcium nitrate, magnesium nitrate, strontium nitrate, and barium nitrate.

Examples of the thiocyanate can include sodium thiocyanate, ammonium thiocyanate, and guanidinium thiocyanate.

Examples of the perchlorate can include ammonium perchlorate, potassium perchlorate, calcium perchlorate, silver perchlorate, sodium perchlorate, and magnesium perchlorate.

These inorganic salts may be used alone or in a combination of two or more thereof.

Examples of a preferred inorganic salt include an alkali metal halide and an alkaline earth metal halide. Specific examples of the preferred inorganic salt can include lithium chloride and calcium chloride.

An addition amount (content) of the inorganic salt may be 0.5% by mass to 10% by mass or 0.5% by mass to 5% by mass, with respect to a total mass of the solvent.

An insoluble matter may be removed from the protein solution, if necessary. That is, the method for producing a raw material molded article may include a step of removing an insoluble matter from the protein solution after the dissolution step, if necessary. Examples of a method for removing the insoluble matter from the protein solution include general methods such as centrifugation and filter filtration with a drum filter, a press filter, or the like. In the case of filter filtration, the insoluble matter can be more efficiently removed from the protein solution using a filter aid such as celite or diatomaceous earth and a pre-coating agent in combination.

The protein solution contains a protein and a solvent (solvent for dissolution) that dissolves the protein. The protein solution may contain impurities included together with the protein in the dissolution step. The protein solution may be a solution for molding a protein molded article.

A content of the protein in the protein solution may be 5% by mass to 35% by mass or 5% by mass to 50% by mass, with respect to a total amount of the protein solution.

A method for producing the protein in which a hydroxyl group is esterified is not particularly limited, and a method of producing the protein using a carboxylic acid such as formic acid as a solvent in the above-described dissolution step may be used, or a method other than the method of producing the protein in the above-described dissolution step may be used. Such a method may be, for example, a method of producing the protein in a step of reacting at least one selected from the group consisting of a carboxylic acid, acid anhydride, and acid chloride with a protein having a hydroxyl group such as serine, tyrosine, or threonine.

### [Molding Step]

A molding step is a step of molding a raw material molded article using a protein solution. A form of the raw material molded article is not particularly limited, and examples thereof can include a fiber, a heat compression molded article, a film, a porous body, a gel, and a resin.

In the protein solution, it is preferable to adjust a concentration and viscosity of the protein depending on the raw material molded article to be molded.

A method for adjusting the concentration of the protein in the protein solution is not particularly limited, and examples thereof include a method of increasing the concentration of the protein by evaporating a solvent by distillation, a method using a solution having a high concentration of the protein in the dissolution step, and a method of reducing an addition amount of a solvent with respect to the amount of the protein.

A viscosity suitable for spinning is generally 10 to 50,000 cP (centipoise), and the viscosity can be measured using, for example, an “EMS viscometer” (trade name) manufactured by Kyoto Electronics Manufacturing Co., Ltd. When the viscosity of the protein solution is not within a range of 10 to 10,000 cP (centipoise), the viscosity of the protein solution may be adjusted to a viscosity at which spinning can be performed. The viscosity can be adjusted using the above-described method and the like. The solvent may contain an appropriate inorganic salt as exemplified above.

In a case where the raw material molded article to be molded is a fiber (raw material fiber), a content (concentration) of the protein in the protein solution may be adjusted to have a concentration and viscosity at which spinning can be performed, if necessary. A method for adjusting the concentration and viscosity of the protein is not particularly limited. In addition, examples of a spinning method include wet spinning. When the protein solution having the adjusted concentration and viscosity suitable for spinning is added to a coagulation liquid as a dope solution, the protein coagulates. In this case, since the protein solution is added to the coagulation liquid as a yarn-shaped liquid, the protein coagulates in a yarn state, and thus, a yarn (undrawn yarn) can be formed. The undrawn yarn can be formed, for example, according to the method disclosed in JP 5584932 B2.

Hereinafter, an example of the wet spinning will be described, but the spinning method is not particularly limited, and may be dry wet spinning.

Wet Spinning and Drawing

(a) Wet Spinning

The coagulation liquid may be any solution that can be desolventized. As the coagulation liquid, it is preferable to use a lower alcohol having 1 to 5 carbon atoms, such as methanol, ethanol, and 2-propanol, or acetone. The coagulation liquid may also contain water. A temperature of the coagulation liquid is preferably 5 to 30° C. from the viewpoint of stability of the spinning.

A method for adding the protein solution as a yarn-shaped liquid is not particularly limited, and examples thereof include a method of extruding the protein solution from a spinneret for spinning to a coagulation liquid in a desolvation bath. An undrawn yarn is obtained by coagulating the protein. An extrusion speed in a case where the protein solution is extruded to the coagulation liquid can be appropriately set according to a diameter of the spinneret, a viscosity of the protein solution, or the like. For example, in a case of a syringe pump having a nozzle with a diameter of 0.1 to 0.6 mm, an extrusion speed is preferably 0.2 to 6.0 mL/h per hole and more preferably 1.4 to 4.0 mL/h per hole, from the viewpoint of the stability of the spinning. A length of the desolvation bath (coagulation liquid bath) into which the coagulation liquid is introduced is not particularly limited, and may be, for example, 200 to 500 mm. A take-up speed of the undrawn yarn formed by the coagulation of the protein may be, for example, 1 to 14 m/min, and retention time may be, for example, 0.01 to 0.15 min. The take-up speed of the undrawn yarn is preferably 1 to 3 m/min from the viewpoint of efficiency of desolvation. The undrawn yarn formed by the coagulation of the protein may be further drawn (pre-drawn) in a coagulation liquid, however, it is preferable that the coagulation liquid is kept at a low temperature and the undrawn yarn is taken up from the coagulation liquid in a form of an undrawn yarn, from the viewpoint of suppressing evaporation of the lower alcohol used in the coagulation liquid.

(b) Drawing

A step of further drawing the undrawn yarn obtained by the above-described method can be included. The drawing may be one-stage drawing or multi-stage drawing including two or more stages. When the drawing is performed in multi stages, molecules can be aligned in multiple stages and a total draw ratio can be increased, which is suitable for producing a fiber having high toughness.

The raw material fiber may be subjected to a shrink-proofing treatment in the wet spinning. Examples of a method for shrink-proofing the raw material fiber can include a method of bringing a protein fiber before contact with water into contact with water after spinning so as to irreversibly shrink the fiber (water shrinking method) and a method of heating a protein fiber before contact with water after spinning and relaxing the protein fiber in the heated state so as to irreversibly shrink the fiber (dry heat shrinking method).

It is considered that the irreversible shrinkage of the protein fiber occurs, for example, due to the following reasons. That is, a secondary structure or a tertiary structure of the protein fiber is considered as one reason for the occurrence of the irreversible shrinkage. Furthermore, in the protein fiber having a residual stress caused by the drawing performed during the production process, the residual stress is relaxed, which is considered as another reason for the occurrence of the irreversible shrinkage.

The water shrinking method includes a step of bringing a protein fiber before contact with water into contact with water after spinning so as to irreversibly shrink the fiber (shrinking step). In the shrinking step, the protein fiber shrinks when being brought into contact with water regardless of an external force. The water to be brought into contact may be water in ether a liquid state or a gas state. A method for bringing the protein fiber into contact with water is not particularly limited, and examples thereof include a method of immersing the protein fiber in water, a method of spraying water at room temperature or steam of heated water onto the protein fiber, and a method of exposing the protein fiber to a high-humidity environment filled with water vapor. Among these methods, the method of immersing the protein fiber in water is preferred, since the shrinkage time can be effectively shortened, and the processing equipment can be simplified. Specific examples of the method of immersing the protein fiber in water include a method of bringing the protein fiber into contact with water by introducing the protein fiber into a container containing water at a predetermined temperature.

A temperature of the water brought into contact with the protein fiber is not particularly limited, but is preferably, for example, lower than the boiling point. At such a temperature, handleability, workability in the shrinking step, and the like are improved. Furthermore, an upper limit of the temperature of the water is preferably 90° C. or lower and more preferably 80° C. or lower. A lower limit of the temperature of the water is preferably 10° C. or higher, more preferably 40° C. or higher, and even more preferably 70° C. or higher. The temperature of the water brought into contact with the protein fiber can be adjusted according to a fiber constituting the protein fiber. In addition, while the water is brought into contact with the protein fiber, the temperature of the water may be constant or changed to a predetermined temperature.

Time for the contact between the protein fiber and water is not particularly limited, and may be, for example, 1 minute or longer. The time may be 10 minutes or longer, 20 minutes or longer, or 30 minutes or longer. In addition, an upper limit of the time is not particularly limited, and may be, for example, 120 minutes or shorter, 90 minutes or shorter, or 60 minutes or shorter, from the viewpoints of shortening the time of the production process and eliminating the possibility of hydrolysis of the protein fiber.

The water shrinking method may further include, following the shrinking step, a step of drying (drying step) after brining the protein fiber into contact with water.

A drying method in the drying step is not particularly limited, and the drying may be, for example, natural drying or forced drying using drying equipment. A drying temperature is not limited as long as it is a temperature lower than a temperature at which the protein is thermally damaged, and in general, may be a temperature within a range of 20 to 150° C. The drying temperature is preferably a temperature within a range of 40 to 120° C. and more preferably a temperature within a range of 60 to 100° C. When the temperature is within the above range, the protein fiber can be more quickly and efficiently dried without thermally damaging the protein or the like. Drying time is appropriately selected according to the drying temperature or the like, and for example, time during which the influence of overdrying of the protein fiber on the quality and physical properties of a knitted fabric can be eliminated is employed.

The dry heat shrinking method includes a step of heating the protein fiber before the contact with water after spinning (heating step) and a step of relaxing the protein fiber in the heated state so as to irreversibly shrink the protein fiber (relaxation and shrinking step).

In the heating step, a heating temperature is preferably a softening temperature of the protein used in the protein fiber or higher. As used herein, the softening temperature of the protein is a temperature at which shrinkage of the protein fiber is initiated due to stress relaxation. In the heat relaxation and shrinking at the softening temperature of the protein or higher, a fiber shrinks to the extent that cannot be achieved by simply removing water from the fiber. As a result, in the obtained protein fiber, shrinkage by contact with water, that is, dimensional change is sufficiently suppressed. The heating temperature is preferably 80° C. or higher, more preferably 180° C. to 280° C., even more preferably 200° C. to 240° C., and still more preferably 220° C. to 240° C.

Heating time in the heating step is preferably 60 seconds or shorter, more preferably 30 seconds or shorter, and even more preferably 5 seconds or shorter, from the viewpoint of a degree of elongation of the fiber after the heat treatment. It is considered that the length of the heating time does not significantly affect the stress.

In the relaxation and shrinking step, the relaxation ratio is preferably greater than 1 time, more preferably 1.4 times or greater, even more preferably 1.7 times or greater, and particularly preferably 2 times or greater. The relaxation ratio is determined as, for example, a ratio of a delivery speed to a winding speed of the protein fiber.

In a case where the raw material molded article is a heat compression molded article (raw material heat compression molded article), a method for forming the raw material heat compression molded article is not particularly limited. For example, a dried protein powder is introduced into a compression molding machine, and pressurization and heating are performed using a hand press machine or the like, such that the dried protein powder reaches a required temperature, and thus, a heat compression molded article can be obtained. Furthermore, the raw material heat compression molded article can be formed according to a method disclosed in patent literature (Japanese Patent Application No. 2017-539869 and PCT/JP 2016/076500).

In a case where the raw material molded article is a film (raw material film), the concentration and viscosity of the protein solution may be adjusted so that the protein solution can be formed into a film, if necessary. A method for forming a protein into a film is not particularly limited, and examples thereof include a method of obtaining a film having a predetermined thickness by applying a protein solution onto a flat plate having a resistance to a solvent in a predetermined thickness to form a coating film, and removing the solvent from the coating film.

Examples of a method for forming a film having a predetermined thickness include a casting method. In a case where the film is formed by a casting method, a protein film (polypeptide film) can be obtained by casting, on a flat plate, the protein solution to a thickness of several microns or more using a tool such as a doctor coat or a knife coater to form a cast film, and then removing the solvent by reduced pressure drying or immersion in a desolvation bath. The raw material film can be formed according to the method disclosed in JP 5678283 B2.

In a case where the raw material molded article is a porous body (raw material porous body), the concentration and viscosity may be adjusted so that the porous body can be formed, if necessary. A method for forming the raw material porous body is not particularly limited. Examples of the method include a method of obtaining a porous body by adding an appropriate amount of a foaming agent to the protein solution of which a concentration and viscosity are adjusted to be suitable for forming the protein solution into a porous body and removing the solvent, and the method described in JP 5796147 B2.

In a case where the raw material molded article is a gel (raw material gel), a method for forming the raw material gel is not particularly limited. For example, the raw material gel can be obtained by a solution production step of dissolving a dry protein in a solvent for dissolution to obtain a solution of a polypeptide and a step of substituting the solution produced in the solution production step with a water-soluble solvent. In this case, a step of pouring the solution into a mold to mold the raw material gel into a predetermined shape is performed between the solution production step and the step of substituting the solvent for dissolution with the water-soluble solvent, or cutting of the raw material gel into a predetermined shape can be performed after the step of substituting the solvent for dissolution with the water-soluble solvent. In addition, the raw material gel can be formed, for example, according to the method disclosed in JP 5782580 B2.

In a case where the raw material molded article is a resin (raw material resin), the concentration and viscosity of the protein solution may be adjusted so that the protein solution can be formed into a resin, if necessary. A method for forming the raw material resin is not particularly limited, and the raw material resin can be produced according to the method disclosed in JP 5678283 B2.

[Method for Processing Protein Molded Article]

A method for processing a protein molded article according to the present embodiment includes a step of bringing a protein molded article containing a protein in which a hydroxyl group is esterified into contact with an acidic or basic medium in a state of applying a tensile force, thus hydrolyzing an ester group.

As for the protein in which a hydroxyl group is esterified, the amount of the tensile force, a method for applying the tensile force, properties of the acidic or basic medium, and a method for hydrolyzing an ester group in the present embodiment, the same aspects as those of the above-described method for producing a protein molded article can be applied. The protein molded article may be obtained by a method including the step of hydrolyzing an ester group (hydrolysis step) in the above-described method for producing a protein molded article, or may be obtained by a method not including the hydrolysis step.

According to the processing method according to the present embodiment, the hydroxyl group in the protein can be exposed again, and thus, it is possible to provide a method for processing a protein molded article which can solve a problem caused by the esterification of a hydroxyl group contained in a protein while maintaining a sufficient strength.

### EXAMPLES

Hereinafter, the present invention will be described more specifically with reference to Examples, however, the present invention is not limited to these Examples.

[Production of Protein]

(1) Preparation of Expression Vector

Spider silk fibroin (hereinafter, also referred to as “PRT966”) having SEQ ID NO: 40 was designed based on the base sequence and the amino acid sequence of Nephila clavipes-derived fibroin (GenBank accession number: P46804.1, GI: 1174415).

Next, a nucleic acid encoding the designed protein (spider silk fibroin) was synthesized. To the nucleic acid, an NdeI site was added at the 5′-end, and an EcoRI site was added downstream of the stop codon. The nucleic acid was cloned into a cloning vector (pUC118). Thereafter, the nucleic acid was cleaved at NdeI and EcoRI by restriction enzyme treatment and then recombined with a protein expression vector pET-22b(+) to obtain an expression vector.

(2) Expression of Protein

Escherichia coli BLR(DE3) was transformed with the expression vector obtained in (1). The transformed Escherichia coli was cultured in 2 mL of LB medium containing ampicillin for 15 hours. The culture solution was added to 100 mL of a medium for seed culture containing ampicillin (Table 4) so that OD₆₀₀ reached 0.005. Flask culture was performed to an OD₆₀₀ of 5 (for approximately 15 hours) while maintaining the culture solution temperature at 30° C., to obtain a seed culture solution.

TABLE 4
Medium for seed culture

| Reagent | Concentration (g/L) |
|---|---|
| Glucose | 5.0 |
| KH₂PO₄ | 4.0 |
| K₂HPO₄ | 9.3 |
| Yeast Extract | 6.0 |
| Ampicillin | 0.1 |

The seed culture solution was added to a jar fermenter to which 500 mL of a production medium (Table 5) was added so that OD₆₀₀ reached 0.05. Culture was performed while maintaining the culture solution temperature at 37° C. and controlling the pH to be constant at 6.9. In addition, the dissolved oxygen concentration in the culture solution was maintained at 20% of the dissolved oxygen saturation concentration.

TABLE 5
Production Medium

| Reagent | Concentration (g/L) |
|---|---|
| Glucose | 12.0 |
| KH₂PO₄ | 9.0 |
| MgSO₄•7H₂O | 2.4 |
| Yeast Extract | 15 |
| FeSO₄•7H₂O | 0.04 |
| MnSO₄•5H₂O | 0.04 |
| CaCl₂•2H₄O | 0.04 |
| GD-113 (antifoaming agent) | 0.1 (mL/L) |

Immediately after glucose in the production medium was completely consumed, a feed solution (455 g/1 L glucose and 120 g/1 L yeast extract) was added at a speed of 1 mL/min. Culture was performed while maintaining the culture solution temperature at 37° C. and controlling the pH to be constant at 6.9. The dissolved oxygen concentration in the culture solution was also maintained at 20% of the dissolved oxygen saturation concentration, and the culture was performed for 20 hours. The expression of the modified fibroin was then induced by adding 1 M isopropyl-β-thiogalactopyranoside (IPTG) to the culture solution so that the final concentration was 1 mM. 20 hours after the addition of IPTG, the bacterial cells were collected by centrifuging the culture solution. SDS-PAGE was performed by using the bacterial cells prepared from the culture solution before the addition of IPTG and the culture solution after the addition of IPTG, and expression of the target protein (spider silk fibroin) was confirmed by appearance of a band of the target recombinant protein depending on the addition of IPTG.

(3) Purification of Protein

Bacterial cells that were collected two hours after the addition of IPTG were washed with 20 mM Tris-HCl buffer (pH 7.4). The washed bacterial cells were suspended in a 20 mM Tris-HCl buffer solution (pH 7.4) containing about 1 mM PMSF, and the cells were disrupted with a high-pressure homogenizer (manufactured by GEA Niro Soavi). The disrupted cells were centrifuged, thus obtaining a precipitate. The obtained precipitate was washed with a 20 mM Tris-HCl buffer solution (pH 7.4) until the purity of the precipitate became high. The washed precipitate was suspended in an 8 M guanidine buffer solution (8 M guanidine hydrochloride, 10 mM sodium dihydrogen phosphate, 20 mM NaCl, and 1 mM Tris-HCl, pH 7.0) so that the concentration became 100 mg/mL, and dissolved by stirring with a stirrer at 60° C. for 30 minutes. After the dissolution, dialysis was performed with water using a dialysis tube (cellulose tube 36/32 manufactured by Sanko Junyaku Co., Ltd.). A white aggregate protein obtained after the dialysis was collected by centrifugation, moisture was removed with a lyophilizer, and the lyophilized powder was collected, thereby obtaining a protein (spider silk fibroin).

(4) Production of Raw Material Fibers

The dry powder of spider silk fibroin was dissolved in formic acid, and filtration was performed with a metal filter having a mesh size of 1 μm, thereby obtaining a dope solution (the concentration of the protein in the dope solution: 30% by mass). The dope solution was discharged to a coagulation liquid by a gear pump using a known spinning apparatus. Spinning conditions were as follows. As a result, raw material fibers (raw material spider silk fibroin fibers) were obtained. (Spinning conditions) Nozzle hole diameter: 0.1 mm; Coagulation liquid: methanol; Temperature of coagulation liquid: 25° C.; Temperature of water washing bath: 25° C.; Hot roller temperature: 60° C.

(5) Hydrolysis Treatment

The raw material fibers obtained in (4) were subjected to a hydrolysis treatment by the following steps [i] to [iii].

[i] The raw material fibers were immersed and stirred in an alkaline aqueous solution.
[ii] The raw material fibers were washed with tap water until the pH became 8.0 or lower.
[iii] The raw material fibers were naturally dried for 2 days.

Table 6 shows treatment conditions in the step [i]. In Example 1, the raw material fibers were wound (winding width: 20 cm, number of layers: 20 plies) on a stainless-steel plate having a size of 300×300×2 (mm) while applying tension (load: 10 N) using a filament winder FWM-1500LF (manufactured by Asahi Kasei Engineering Corporation)

TABLE 6

| | Temper- | Treatment | | |
|---|---|---|---|---|
| | ature | time | Treatment | |
| | [° C.] | [min] | medium | Tension |
| Example 1 | 25 | 50 | Na₂CO₃ aqueous | Applied |
| (n = 4) | | | solution with pH | |
| | | | of 11.4 to 11.5 | |
| Comparative | 25 | 50 | Na₂CO₃ aqueous | Not |
| Example 1 | | | solution with pH | applied |
| | | | of 11.4 to 11.5 | |

(6) Evaluation of Formyl Residue (Formic Acid Ester Group) Removal by FT-IR

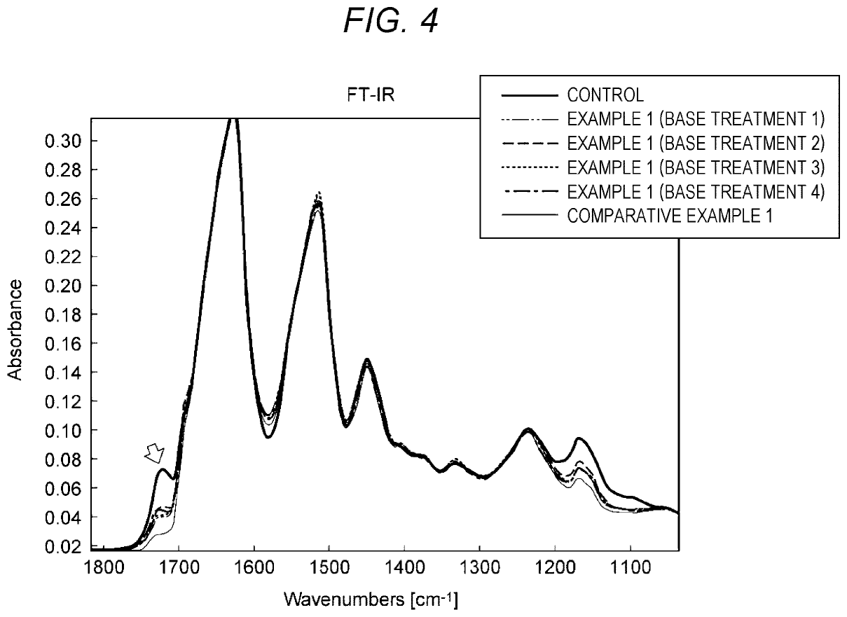

Each of the fibers obtained in (5) was subjected to evaluation of formyl residue removal by performing measurement by the ATR method (total reflectance method) using a Fourier transform infrared spectrometer (Nicolet iS50FT-IR, manufactured by Thermo Fisher Scientific Inc.). For each of the fibers (Example 1 (n=4) and Comparative Example 1), a height of a peak at a wave number of 1,730 cm⁻¹ (peak attributable to C═O of the formyl residue, the arrow in FIG. 4) was confirmed from an IR spectrum to evaluate whether or not the peak was detected (FIG. 4). In a case where the peak was not detected, it was determined that the formyl residue was removed. A fiber on which the (5) hydrolysis treatment described above was not performed was used as a control.

From FIG. 4, it was confirmed that a hydrolysis reaction of the formyl residue proceeded when the raw material fiber was subjected to the hydrolysis treatment with the alkaline aqueous solution, thus removing the formyl residue in the protein fiber (Example 1 and Comparative Example 1).

In the FT-IR spectrum diagram (FIG. 4), peaks were fit using a Lorentzian function, and a peak of which the intensity did not change before and after the hydrolysis treatment (near 1,470 cm⁻¹) was selected as a base peak. A ratio of an area of a peak attributable to a formic acid ester to an area of the base peak (area ratio) was obtained. A ratio [%] of the formyl residue in Example 1 was calculated by obtaining an area ratio in Example 1 with respect to an area ratio in the control. The results are shown in Table 7.

TABLE 7

| | | | Formyl |
|---|---|---|---|
| | | Area ratio | residues [%] |
| Control | | 0.216 | 100 |
| Example 1 | Base treatment 1 | 0.045 | 20.83 |
| (n = 4) | Base treatment 2 | 0.074 | 34.26 |
| | Base treatment 3 | 0.051 | 23.61 |
| | Base treatment 4 | 0.071 | 32.87 |
| | Average of base | — | 27.89 |
| | treatments 1 to 4 | | |

Assuming that all hydroxyl groups were formylated before the treatment, a portion that was not deformylated can be calculated to be 0.52 mmol/g by multiplying the amount of hydroxyl groups in the spider silk fibroin (1.88 mmol/g) by the ratio of the formyl residue (27.89%). Using this calculation result, a deformylated portion can be calculated to be 1.36 mmol/g (=1.88 mmol/g−0.52 mmol/g).

(7) Evaluation of Degree of Elongation

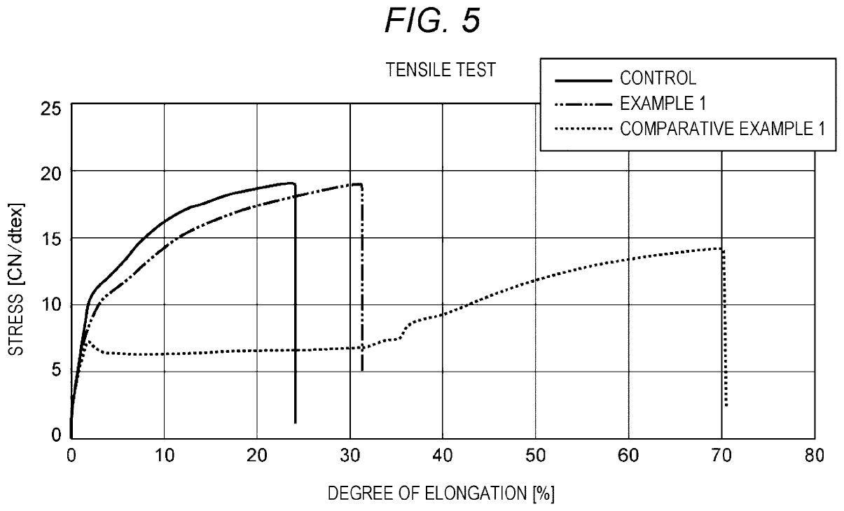

Each of the fibers that has been subjected to the hydrolysis treatment and dried was fixed on a gripping tool at a fiber length of 20 mm, and a stress (strength) and a degree of elongation were measured at a tensile speed of 10 mm/min using a single-fiber tensile tester FAVIMAT+ manufactured by Textechno H. Stein GmbH & Co. KG under conditions of a load cell of 2 N capacity, pretension of 1.25 cmN/tex, a temperature of 20° C., and a relative humidity of 65%. The results are shown in FIG. 5.

When Example 1 and Comparative Example 1 were compared with each other, the strength of the protein fiber was shown to be improved by applying a tensile force during the hydrolysis treatment. In addition, when the control and Example 1 were compared with each other, the degree of elongation was increased, and toughness of the protein fiber (an area of a region surrounded by the stress (strength) and the degree of elongation) was shown to be improved by applying the tensile force during the hydrolysis treatment.

1. A method for producing a protein molded article comprising: a step of bringing a raw material molded article containing a protein in which a hydroxyl group is esterified into contact with an acidic or basic medium in a state of applying a tensile force, thus hydrolyzing an ester group.

2. The method for producing a protein molded article according to claim 1, wherein the medium is an aqueous solution.

3. The method for producing a protein molded article according to claim 2, wherein the aqueous solution is an alkaline aqueous solution with a pH lower than 12.

4. The method for producing a protein molded article according to claim 2, wherein the amount of the tensile force is an amount in which the raw material molded article does not shrink by the contact with the aqueous solution.

5. The method for producing a protein molded article according to claim 1, wherein the protein is a structural protein.

6. The method for producing a protein molded article according to claim 5, wherein the structural protein is fibroin.

7. The method for producing a protein molded article according to claim 6, wherein the fibroin is spider silk fibroin.

8. The method for producing a protein molded article according to claim 1, wherein the protein in which the hydroxyl group is esterified contains a formic acid ester.

9. The method for producing a protein molded article according to claim 1, wherein the raw material molded article is at least one selected from the group consisting of a fiber, a heat compression molded article, a film, a porous body, a gel, and a resin.

10. A method for processing a protein molded article comprising: a step of bringing a protein molded article containing a protein in which a hydroxyl group is esterified into contact with an acidic or basic medium in a state of applying a tensile force, thus hydrolyzing an ester group.
